(19) United States
(12) Patent Application Publication
(10) Pub. No.: US 2008/0262035 A1
(43) Pub. Date: Oct. 23, 2008

(54) Amide Derivatives as Inhibitors of Histone Deacetylase

(76) Inventors: Prasun K. Chakravarty, Rahway, NJ (US); Steven L. Colletti, Rahway, NJ (US); Raffaele Ingenito, Rome (IT); Peter T. Meinke, Rahway, NJ (US); Alessia Petrocchi, Rome (IT); Christian Steinkuhler, Rome (IT)

Correspondence Address: MERCK AND CO., INC, P O BOX 2000, RAHWAY, NJ, 07065-0907, US

(21) Appl. No.: 11/632,235

(22) PCT Filed: Jul. 11, 2005
(86) PCT No.: PCT/GB2005/002752

§ 371 Date: Jan. 22, 2008

Related U.S. Application Data
(60) Provisional application No. 60/587,180, filed on Jul. 12, 2004.

(51) Int. Cl. A61K 31/4709; C07D 209/04; A61K 31/404; A61P 25/00; A61P 37/00; A61P 35/00; C07D 215/00

(52) U.S. Cl. 514/313; 548/455; 514/414; 548/495; 514/419; 546/162

(57) ABSTRACT

The present invention relates to carboxylic acid derivatives that are inhibitors of histone deacetylase (HDAC). The compounds of the present invention are useful for treating cellular proliferative diseases, including cancer. Further, the compounds of the present invention are useful for treating neurodegenerative diseases, schizophrenia and stroke among other diseases.

### BACKGROUND OF THE INVENTION

DNA in the nucleus of the cell exists as a hierarchy of compacted chromatin structures. The basic repeating unit in chromatin is the nucleosome. The nucleosome consists of a histone octamer of proteins in the nucleus of the cell around which DNA is wrapped twice. The orderly packaging of DNA in the nucleus plays an important role in the functional aspects of gene regulation. Covalent modifications of the histones have a key role in altering chromatin higher order structure and function and ultimately gene expression. The covalent modification of histones, such as acetylation, occurs by enzymatically mediated processes.

Regulation of gene expression through the inhibition of the nuclear enzyme histone deacetylase (HDAC) is one of several possible regulatory mechanisms whereby chromatin activity can be affected. The dynamic homeostasis of the nuclear acetylation of histones can be regulated by the opposing activity of the enzymes histone acetyl transferase (HAT) and histone deacetylase (HDAC). Transcriptionally silent chromatin can be characterized by nucleosomes with low levels of acetylated histones. Acetylation reduces the positive charge of histones, thereby expanding the structure of the nucleosome and facilitating the interaction of transcription factors with the DNA. Removal of the acetyl group restores the positive charge, condensing the structure of the nucleosome. Histone acetylation can activate DNA transcription, enhancing gene expression. Histone deacetylase can reverse the process and can serve to repress gene expression. See, for example, Grunstein, Nature 389, 349-352 (1997); Pazin et al., Cell 89, 325-328 (1997); Wade et al., Trends Biochem. Sci. 22, 128-132 (1997); and Wolffe, Science 272, 371-372 (1996).

WO 01/18171 and WO 2005/051901 describe HDAC inhibitors as cancer agents.

### SUMMARY OF THE INVENTION

The present invention relates to carboxylic acid derivatives that are inhibitors of histone deacetylase (HDAC). The compounds of the present invention are useful for treating cellular proliferative diseases, including cancer. Further, the compounds of the present invention are useful for treating neurodegenerative diseases, schizophrenia and stroke among other diseases.

### DETAILED DESCRIPTION OF THE INVENTION

The compounds of this invention are useful in the inhibition of histone deacetylase. A first embodiment of the instant invention is a compound as illustrated by Formula I:

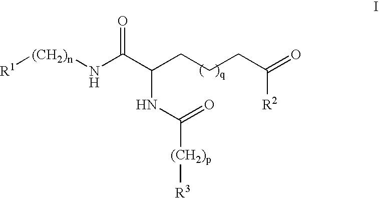

wherein:

a is 0 or 1; b is 0 or 1; m is 0, 1 or 2; n is 0, 1, 2 or 3; p is 0, 1, 2 or 3; and q is 1, 2, 3 or 4;

R¹ is selected from: (C═O)ₐO_{b}(C₁-C₆)alkyl, NH(C═O)(C₁-C₆)alkyl, N(R^{c})₂, (O)ₐ-aryl, (C₃-C₈)cycloalkyl, and heterocyclyl; said alkyl, cycloalkyl, aryl and heterocyclyl optionally substituted with up to three substituents selected from R^{d};

R² is selected from: OH, O(C₁-C₆)alkyl and N(R^{b})₂;

R³ is selected from: H, CF₃, oxo, OH, halogen, CN, N(R^{c})₂, NO₂, (C═O)ₐO_{b}(C₁-C₁₀)alkyl, (C═O)ₐO_{b}(C₂-C₁₀)alkenyl, (C═O)ₐO_{b}(C₂-C₁₀)alkynyl, (C═O)ₐO_{b}(C₃-C₈)cycloalkyl, (C═O)ₐO_{b}(C₁-C₆)alkylene-aryl, (C═O)ₐO_{b}-aryl, (C═O)ₐO_{b}(C₁-C₆)alkylene-heterocyclyl, (C═O)ₐO_{b}-heterocyclyl, NH(C═O)ₐ-aryl, (C₁-C₆)alkyl(O)ₐ-phenyl, (C═O)ₐO_{b}(C₁-C₆)alkylene-N(R^{a})₂, N(R^{a})₂, O_{b}(C₁-C₃)perfluoroalkyl, (C₁-C₆)alkylene-S(O)ₘR^{a}, S(O)ₘR^{a}, C(O)R^{a}, (C₁-C₆)alkylene-CO₂R^{a}, CO₂R^{a}, C(O)H, C(O)N(R^{a})₂, and S(O)₂N(R^{a})₂; said alkyl, alkenyl, alkynyl, cycloalkyl, phenyl, aryl, alkylene and heterocyclyl is optionally substituted with up to three substituents selected from R^{e};

R^{a} is independently selected from: H, oxo, OH, halogen, CO₂H, CN, (O)C═O(C₁-C₆)alkyl, N(R^{c})₂, (C₁-C₆)alkyl, aryl, heterocyclyl, (C₃-C₈)cycloalkyl, (C═O)O(C₁-C₆)alkyl, C═O(C₁-C₆)alkyl and S(O)₂R^{a}; said alkyl, cycloalkyl, aryl or heterocylyl is optionally substituted with one or more substituents selected from OH, (C₁-C₆)alkyl, (C₁-C₆)alkoxy, halogen, CO₂H, CN, (O)C═O(C₁-C₆)alkyl, oxo, N(R^{c})₂ and optionally substituted heterocyclyl, wherein said heterocyclyl is optionally substituted with (C₁-C₆)alkyl, oxo or NH₂;

R^{b} is independently selected from: H, OH, Oₐ(C₁-C₆)alkyl, N(R^{c})₂ and phenyl; said alkyl and phenyl is optionally substituted with phenyl and N(R^{g})₂;

R^{c} is independently selected from: H, (C═O)ₐO_{b}(C₁-C₆)alkyl-phenyl and (C═O)ₐO_{b}(C₁-C₆)alkyl;

R^{d} is independently selected from: NO₂, Oₐ-aryl, Oₐ-heterocyclyl, NH(C═O)-aryl, NH(C═O)(C₁-C₆)alkyl, (C═O)N(R^{c})₂, Oₐ-perfluoroalkyl, OₐCF₃, (C═O)ₐ(C₁-C₆)alkyl, NHS(O)ₘ-aryl, NHS(O)ₘ(C₁-C₆)alkyl, N(R^{c})₂, Oₐ(C₁-C₆)alkyl-heterocyclyl, Oₐ(C₁-C₆)alkyl-N(R^{g})₂, S(O)ₘ(C₁-C₆)alkyl, S(O)ₘ-aryl, (C═O)ₐ-aryl, Oₐ(C₁-C₆)alkyl, CN, S(O)ₘN(R^{c})₂, oxo, OH and halo; wherein said alkyl, aryl and heterocyclyl are optionally substituted with R^{f};

R^{e} is independently selected from: (C═O)ₐCF₃, oxo, OH, halogen, CN, N(R^{c})₂, NO₂, (C═O)ₐO_{b}(C₁-C₁₀)alkyl, (C═O)ₐO_{b}(C₂-C₁₀)alkenyl, (C═O)ₐO_{b}(C₂-C₁₀)alkynyl, (C═O)ₐO_{b}(C₃-C₈)cycloalkyl, (C═O)ₐO_{b}(C₁-C₆)alkylene-aryl, (C═O)ₐO_{b}-aryl, (C═O)ₐO_{b}(C₁-C₆)alkylene-heterocyclyl, (C═O)ₐO_{b}-heterocyclyl, NH(C═O)ₐ-aryl, (C₁-C₆)alkyl(O)ₐ-phenyl, (C═O)ₐO_{b}(C₁-C₆)alkylene-N(R^{a})₂, N(R^{a})₂, O_{b}(C₁-C₃)perfluoroalkyl, (C₁-C₆)alkylene-S(O)ₘR^{a}, S(O)ₘR^{a}, C(O)R^{a}, (C₁-C₆)alkylene-CO₂R^{a}, CO₂R^{a}, C(O)H, (C₁-C₆)alkylₐNH(C₁-C₆)alkyl-N(R^{c})₂, C(O)N(R^{a})₂, and S(O)₂N(R^{a})₂;

R^{f} is independently selected from phenyl, heterocyclyl and Oₐ(C₁-C₆)alkyl;

R^{g} is independently selected from H and (C₁-C₆)alkyl;

or a pharmaceutically acceptable salt or stereoisomer thereof.

A second embodiment of the instant invention is a compound as illustrated by Formula II;

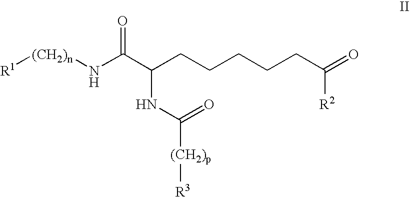

wherein:

all substituents and variables are as defined above;

or a pharmaceutically acceptable salt or stereoisomer thereof.

A third embodiment of the instant invention is a compound as illustrated by Formula II;

wherein:

R³ is selected from: H, CN, CF₃, N(R^{c})₂, (C₂-C₁₀)alkenyl, (C₃-C₈)cycloalkyl, S(O)₂(C₁-C₆)alkyl, (C═O)ₐO_{b}(C₁-C₁₀)alkyl, (C═O)ₐ-aryl, (C═O)ₐ— heterocyclyl, S-aryl, S-heterocyclyl, NH(C═O)ₐ-aryl, (C₁-C₆)alkyl(O)ₐ-phenyl; said alkyl, alkenyl, cycloalkyl, aryl and heterocyclyl is optionally substituted with up to three substituents selected from R^{e};

R^{d} is independently selected from: (C═O)ₐ-phenyl, (C₁-C₆)alkylₐ-heterocyclyl, Oₐ(C₁-C₆)alkyl, oxo, CN, S(O)ₘN(R^{c})₂, OH and halo; wherein said alkyl, phenyl and heterocyclyl are optionally substituted with R^{f};

R^{e} is independently selected from: (C═O)ₐ—CF₃, oxo, OH, halogen, CN, N(R^{c})₂, S(O)₂(C₁-C₆)alkyl, (C₁-C₆)alkylₐ(C═O)NH(C₁-C₆)alkyl-N(R^{c})₂, O(C₁-C₆)alkyl-N(R^{c})₂, (C═O)ₐO_{b}(C₁-C₁₀)alkyl, (C₁-C₆)alkyl-phenyl, aryl, heterocyclyl and S(O)₂-phenyl;

and all substituents and variables are as defined in the second embodiment;

or a pharmaceutically acceptable salt or stereoisomer thereof.

Another embodiment of the instant invention is the use of a compound as illustrated by Formula I:

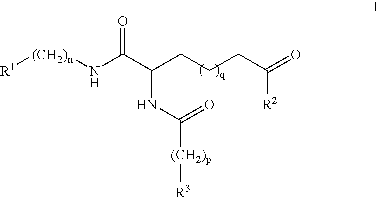

wherein:

a is 0 or 1; b is 0 or 1; m is 0, 1 or 2; n is 0, 1, 2 or 3; p is 0, 1, 2 or 3; and q is 1, 2, 3 or 4;

R¹ is selected from: (C═O)ₐO_{b}(C₁-C₆)alkyl, NH(C═O)(C₁-C₆)alkyl, N(R^{c})₂, (O)ₐ-aryl, (C₃-C₈)cycloalkyl, and heterocyclyl; said alkyl, cycloalkyl, aryl and heterocyclyl optionally substituted with up to three substituents selected from R^{d};

R² is selected from: OH, O(C₁-C₆)alkyl and N(R^{b})₂;

R³ is selected from: H, CF₃, oxo, OH, halogen, CN, N(R^{c})₂, NO₂, (C═O)ₐO_{b}(C₁-C₁₀)alkyl, (C═O)ₐO_{b}(C₂-C₁₀)alkenyl, (C═O)ₐO_{b}(C₂-C₁₀)alkynyl, (C═O)ₐO_{b}(C₃-C₈)cycloalkyl, (C═O)ₐO_{b}(C₁-C₆)alkylene-aryl, (C═O)ₐO_{b}-aryl, (C═O)ₐO_{b}(C₁-C₆)alkylene-heterocyclyl, (C═O)ₐO_{b}-heterocyclyl, NH(C═O)ₐ-aryl, (C₁-C₆)alkyl(O)ₐ-phenyl, (C═O)ₐO_{b}(C₁-C₆)alkylene-N(R^{a})₂, N(R^{a})₂, O_{b}(C₁-C₃)perfluoroalkyl, (C₁-C₆)alkylene-S(O)ₘR^{a}, S(O)ₘR^{a}, C(O)R^{a}, (C₁-C₆)alkylene-CO₂R^{a}, CO₂R^{a}, C(O)H, C(O)N(R^{a})₂, and S(O)₂N(R^{a})₂; said alkyl, alkenyl, alkynyl, cycloalkyl, phenyl, aryl, alkylene and heterocyclyl is optionally substituted with up to three substituents selected from R^{e};

R^{a} is independently selected from: H, oxo, OH, halogen, CO₂H, CN, (O)C═O(C₁-C₆)alkyl, N(R^{c})₂, (C₁-C₆)alkyl, aryl, heterocyclyl, (C₃-C₈)cycloalkyl, (C═O)O(C₁-C₆)alkyl, C═O(C₁-C₆)alkyl and S(O)₂R^{a}; said alkyl, cycloalkyl, aryl or heterocylyl is optionally substituted with one or more substituents selected from OH, (C₁-C₆)alkyl, (C₁-C₆)alkoxy, halogen, CO₂H, CN, (O)C═O(C₁-C₆)alkyl, oxo, N(R^{c})₂ and optionally substituted heterocyclyl, wherein said heterocyclyl is optionally substituted with (C₁-C₆)alkyl, oxo or NH₂;

R^{b} is independently selected from: H, OH, Oₐ(C₁-C₆)alkyl, N(R^{c})₂ and phenyl; said alkyl and phenyl is optionally substituted with phenyl and N(R^{g})₂;

R^{c} is independently selected from: H, (C═O)ₐO_{b}(C₁-C₆)alkyl-phenyl and (C═O)ₐO_{b}(C₁-C₆)alkyl;

R^{d} is independently selected from: NO₂, Oₐ-aryl, Oₐ-heterocyclyl, NH(C═O)-aryl, NH(C═O)(C₁-C₆)alkyl, (C═O)N(R^{c})₂, Oₐ-perfluoroalkyl, OₐCF₃, (C═O)ₐ(C₁-C₆)alkyl, NHS(O)ₘ-aryl, NHS(O)ₘ(C₁-C₆)alkyl, N(R^{c})₂, Oₐ(C₁-C₆)alkyl-heterocyclyl, Oₐ(C₁-C₆)alkyl-N(R^{g})₂, S(O)ₘ(C₁-C₆)alkyl, S(O)ₘ-aryl, (C═O)ₐ-aryl, Oₐ(C₁-C₆)alkyl, CN, S(O)ₘN(R^{c})₂, oxo, OH and halo; wherein said alkyl, aryl and heterocyclyl are optionally substituted with R^{f};

R^{e} is independently selected from: (C═O)ₐCF₃, oxo, OH, halogen, CN, N(R^{c})₂, NO₂, (C═O)ₐO_{b}(C₁-C₁₀)alkyl, (C═O)ₐO_{b}(C₂-C₁₀)alkenyl, (C═O)ₐO_{b}(C₂-C₁₀)alkynyl, (C═O)ₐO_{b}(C₃-C₈)cycloalkyl, (C═O)ₐO_{b}(C₁-C₆)alkylene-aryl, (C═O)ₐO_{b}-aryl, (C═O)ₐO_{b}(C₁-C₆)alkylene-heterocyclyl, (C═O)ₐO_{b}-heterocyclyl, NH(C═O)ₐ-aryl, (C₁-C₆)alkyl(O)ₐ-phenyl, (C═O)ₐO_{b}(C₁-C₆)alkylene-N(R^{a})₂, N(R^{a})₂, O_{b}(C₁-C₃)perfluoroalkyl, (C₁-C₆)alkylene-S(O)ₘR^{a}S(O)ₘR^{a}, C(O)R^{a}, (C₁-C₆)alkylene-CO₂R^{a}, CO₂R^{a}, C(O)H, (C₁-C₆)alkylₐNH(C₁-C₆)alkyl-N(R^{c})₂, C(O)N(R^{a})₂, and S(O)₂N(R^{a})₂;

R^{f} is independently selected from phenyl, heterocyclyl and Oₐ(C₁-C₆)alkyl;

R^{g} is independently selected from H and (C₁-C₆)alkyl;

or a pharmaceutically acceptable salt or stereoisomer thereof, for the manufacture of a medicament for treating or preventing a disease selected from neurodegenerative diseases, schizophrenia, inflammatory diseases, restenosis, mental retardation and immune disorders.

Specific examples of the compounds of the instant invention include:
(7S)-7-{[(5-Methoxy-2-methyl-1H-indol-3-yl)acetyl]amino}-8-oxo-8-{[2-(2-phenyl-1H-indol-3-yl)ethyl]amino}octanoic acid (1);
(2S)-N⁸-(Benzyloxy)-2-{[(5-methoxy-2-methyl-1H-indol-3-yl)acetyl]amino}-N¹-[2-(2-phenyl-1H-indol-3-yl)ethyl]octanediamide (2);
(2S)-N⁸-(2-Aminophenyl)-2-{[(5-methoxy-2-methyl-1H-indol-3-yl)acetyl]amino}-N¹-[2-(2-phenyl-1H-indol-3-yl)ethyl]octanediamide (3);
Methyl (7S)-7-{[(5-methoxy-2-methyl-1H-indol-3-yl)acetyl]amino}-8-oxo-8-{[2-(2-phenyl-1H-indol-3-yl)ethyl]amino}octanoate (4);
(2S)-N⁸-Hydroxy-2-{[(5-methoxy-2-methyl-1H-indol-3-yl)acetyl]amino}-N¹-[2-(2-phenyl-1H-indol-3-yl)ethyl]octanediamide (5);
(2S)-N⁸-Methoxy-2-{[(5-methoxy-2-methyl-1H-indol-3-yl)acetyl]amino}-N⁸-methyl-N¹-[2-(2-phenyl-1H-indol-3-yl)ethyl]octanediamide (6);
(2S)-2-{[(5-Methoxy-2-methyl-1H-indol-3-yl)acetyl]amino}-N¹-[2-(2-phenyl-1H-indol-3-yl)ethyl]octanediamide (7);
(2S)-N⁸-Hydroxy-2-{[(5-methoxy-2-methyl-1H-indol-3-yl)acetyl]amino}-N⁸-methyl-N¹-[2-(2-phenyl-1H-indol-3-yl)ethyl]octanediamide (8);
(2S)-N⁸-Methoxy-2-{[(5-methoxy-2-methyl-1H-indol-3-yl)acetyl]amino}-N¹-[2-(2-phenyl-1H-indol-3-yl)ethyl]octanediamide (9);
(2S)-N⁸-Ethoxy-2-{[(5-methoxy-2-methyl-1H-indol-3-yl)acetyl]amino}-N¹-[2-(2-phenyl-1H-indol-3-yl)ethyl]octanediamide (10);
(2S)-N⁸-(tert-Butoxy)-2-{[(5-methoxy-2-methyl-1H-indol-3-yl)acetyl]amino}-N¹-[2-(2-phenyl-1H-indol-3-yl)ethyl]octanediamide (11);
(2S)-2-{[(5-Methoxy-2-methyl-1H-indol-3-yl)acetyl]amino}-N⁸-methyl-N¹-[2-(2-phenyl-1H-indol-3-yl)ethyl]octanediamide (12);
(2S)-2-{[(5-Methoxy-2-methyl-1H-indol-3-yl)acetyl]amino}-N⁸,N⁸-dimethyl-N¹-[2-(2-phenyl-1H-indol-3-yl)ethyl]octanediamide (13);
(2S)-8-(2,2-Dimethylhydrazino)-2-{[(5-methoxy-2-methyl-1H-indol-3-yl)acetyl]amino}-8-oxo-N-[2-(2-phenyl-1H-indol-3-yl)ethyl]octanamide (14);
(2S)-N⁸-Benzyl-2-{[(5-methoxy-2-methyl-1H-indol-3-yl)acetyl]amino}-N¹-[2-(2-phenyl-1H-indol-3-yl)ethyl]octanediamide (15);
(2S)-2-{[(5-Methoxy-2-methyl-1H-indol-3-yl)acetyl]amino}-N⁸-(2-phenylethyl)-N¹-[2-(2-phenyl-1H-indol-3-yl)ethyl]octanediamide (16);
(2S)-N¹-(4-Chlorophenyl)-N⁸-methoxy-2-{[(5-methoxy-2-methyl-1H-indol-3-yl)acetyl]amino}octanediamide (17);
(2S)-N¹-(4-Chlorophenyl)-N⁸-hydroxy-2-{[(5-methoxy-2-methyl-1H-indol-3-yl)acetyl]amino}-N⁸-methyloctanediamide (18);
(2S)-N⁸-Methoxy-2-{[(5-methoxy-2-methyl-1H-indol-3-yl)acetyl]amino}-N¹-quinolin-3-yloctanediamide (19);
(2S)-N⁸-Methoxy-N¹-[2-(2-phenyl-1H-indol-3-yl)ethyl]-2-[(2-thienylcarbonyl)amino]octanediamide (20);
(2S)-N¹-(4-Chlorophenyl)-N⁸-hydroxy-2-{[(5-methoxy-2-methyl-1H-indol-3-yl)acetyl]amino}octanediamide (21);
(2S)-N⁸-Hydroxy-2-{[(5-methoxy-2-methyl-1H-indol-3-yl)acetyl]amino}-N¹-quinolin-3-yl octanediamide (22);
(2S)-N⁸-Hydroxy-N¹-[2-(2-phenyl-1H-indol-3-yl)ethyl]-2-[(2-thienylcarbonyl)amino]octanediamide (23);
(2S)-N⁸-(2-Aminophenyl)-N¹-(4-chloro phenyl)-2-{[(5-methoxy-2-methyl-1H-indol-3-yl)acetyl]amino}octanediamide (24); and
(8S)-8-{[(5-Methoxy-2-methyl-1H-indol-3-yl)acetyl]amino}-9-oxo-9-{[2-(2-phenyl-1H-indol-3-yl)ethyl]amino}nonanoic acid (25);
   or a pharmaceutically acceptable salt or stereoisomer thereof.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention. In addition, the compounds disclosed herein may exist as tautomers and both tautomeric forms are intended to be encompassed by the scope of the invention, even though only one tautomeric structure is depicted.

When any variable (e.g. R¹ and R², etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on. The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on. In an embodiment of the invention the term “cycloalkyl” includes the groups described immediately above and further includes monocyclic unsaturated aliphatic hydrocarbon groups. For example, “cycloalkyl” as defined in this embodiment includes cyclopropyl, methyl-cyclopropyl, 2,2-dimethyl-cyclobutyl, 2-ethyl-cyclopentyl, cyclohexyl, cyclopentenyl, cyclobutenyl and so on.

The term “alkylene” means a hydrocarbon diradical group having the specified number of carbon atoms. For example, “alkylene” includes —CH₂—, —CH₂CH₂— and the like.

“Alkoxy” represents either a cyclic or non-cyclic alkyl group of indicated number of carbon atoms attached through an oxygen bridge. “Alkoxy” therefore encompasses the definitions of alkyl and cycloalkyl above.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl and biphenyl.

The term “heterocycle” or “heterocyclyl” as used herein is intended to mean a 3- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrahydro analogs thereof. Further examples of “heterocyclyl” include, but are not limited to the following: benzoimidazolyl, benzofurandionyl, benzofuranyl, benzofurazanyl, benzopyrazolyl, benzotriazolyl, benzothiophenyl, benzoxazolyl, carbazolyl, carbolinyl, cinnolinyl, epoxidyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazolinyl, isoxazolinyl, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrothiopyranyl, tetrahydroisoquinolinyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, tetrahydroquinolinyl, dihydroisochromenyl, thiazolidinonyl, imidazolonyl, dihydroimidazolonyl, benzoxazolonyl, benzothiazolyl, isoindolinonyl, octahydroquinolizinyl, octahydroisoindolyl, imidazopyridinyl, azabicycloheptanyl, chromenonyl, dihydrotriazolonyl, benzothiadiazolyl, benzodioxolyl, dihydrobenzodioxinyl, triazolopyrimidinyl dihydroisoindolyl, hydrobenzoxazolyl, azepanyl, oxazolidinyl, azabicycloheptyl and N-oxides thereof. Attachment of a heterocyclyl substituent can occur via a carbon atom or via a heteroatom.

As appreciated by those of skill in the art, “halo” or “halogen” as used herein is intended to include chloro (Cl), fluoro (F), bromo (Br) and iodo (I).

In an embodiment, m is 1 or 2.

In another embodiment, n is 0, 1 or 2.

In another embodiment n is 1, 2 or 3.

In another embodiment, n is 2.

In another embodiment, n is 1.

In another embodiment, n is 0.

In an embodiment p is 0 or 1.

In another embodiment, p is 1.

In an embodiment, q is selected from 2-4.

In another embodiment, q is 3.

In an embodiment, R¹ is selected from: (C═O)ₐO_{b}(C₁-C₆)alkyl, NH(C═O)(C₁-C₆)alkyl, N(R^{c})₂, (O)ₐ-aryl, (C₃-C₈)cycloalkyl, and heterocyclyl; said alkyl, cycloalkyl, aryl and heterocyclyl optionally substituted with up to three substituents selected from R^{d}.

In another embodiment, R¹ is selected from: Oₐ(C₁-C₆)alkyl, NH(C═O)(C₁-C₆)alkyl, N(R^{c})₂, (O)ₐ-phenyl, (C₃-C₈)cycloalkyl, aryl and heterocyclyl; said alkyl, cycloalkyl, phenyl, aryl and heterocyclyl optionally substituted with up to three substituents selected from R^{d}.

Preferably, R¹ is (C₁-C₆)alkyl, O(C₁-C₆)alkyl, N(R^{c})₂ or a ring which is: indolyl, phenyl, isoquinolinyl, imidazopyridinyl, pyrrolidinyl, benzoimidazolyl, cyclopentyl, pyridazinyl, piperidinyl, morpholinyl, furyl, imidazolyl, phenoxy, quinolinyl, thiazolyl, tetrahydronaphthalenyl, dihydroindolyl, pyridinyl, naphthyl, tetrahydrobenzo[7]annulenyl, dihydroindenyl, dihydroisochromenyl, cyclohexyl, benzothiazolyl, isoxazolyl, piperazinyl, cycloheptyl, octahydroquinolizinyl, tetrahydroquinolinyl, biphenyl, benzoxazolyl and thienyl; said alkyl or ring being optionally substituted by up to three substituents selected from R^{d}.

More particularly, R¹ is an optionally substituted phenyl, indolyl or quinolinyl.

In another embodiment, R¹ is selected from: phenyl and heterocyclyl; said phenyl, and heterocyclyl optionally substituted with up to three substituents selected from R^{d}.

In another embodiment, when R¹ is selected from heterocyclyl, said heterocyclyl is selected from:

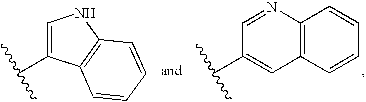

optionally substituted with up to three substituents selected from R^{d}.

In another embodiment, R¹ is

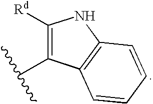

Preferred R¹ groups are phenylindolyl, chlorophenyl and quinolinyl. More particular R¹ groups are 2-phenyl-1H-indol-3-yl, 4-chlorophenyl and quinolin-3-yl.

In an embodiment, R² is selected from: OH, O(C₁-C₆)alkyl and N(R^{b})₂.

Preferably, R² is OH, methoxy or N(R^{b})₂.

In an embodiment, R³ is selected from: H, CF₃, oxo, OH, halogen, CN, N(R^{c})₂, NO₂, (C═O)ₐO_{b}(C₁-C₁₀)alkyl, (C═O)ₐO_{b}(C₂-C₁₀)alkenyl, (C═O)ₐO_{b}(C₂-C₁₀)alkynyl, (C═O)ₐO_{b}(C₃-C₈)cycloalkyl, (C═O)ₐO_{b}(C₁-C₆)alkylene-aryl, (C═O)ₐO_{b}-aryl, (C═O)ₐO_{b}(C₁-C₆)alkylene-heterocyclyl, (C═O)ₐO_{b}-heterocyclyl, NH(C═O)ₐ-aryl, (C₁-C₆)alkyl(O)ₐ-phenyl, (C═O)ₐO_{b}(C₁-C₆)alkylene-N(R^{a})₂, N(R^{a})₂, O_{b}(C₁-C₃)perfluoroalkyl, (C₁-C₆)alkylene-S(O)ₘR^{a}, S(O)ₘR^{a}, C(O)R^{a}, (C₁-C₆)alkylene-CO₂R^{a}, CO₂R^{a}, C(O)H, C(O)N(R^{a})₂, and S(O)₂N(R^{a})₂; said alkyl, alkenyl, alkynyl, cycloalkyl, phenyl, aryl, alkylene and heterocyclyl is optionally substituted with up to three substituents selected from R^{e}.

In another embodiment, R³ is selected from: H, CN, CF₃, N(R^{c})₂, (C₂-C₁₀)alkenyl, (C₃-C₈)cycloalkyl, S(O)₂(C₁-C₆)alkyl, (C═O)ₐO_{b}(C₁-C₁₀)alkyl, (C═O)ₐ-aryl, (C═O)ₐ-heterocyclyl, S-aryl, S-heterocyclyl, NH(C═O)ₐ-aryl, (C₁-C₆)alkyl(O)ₐ-phenyl; said alkyl, alkenyl, cycloalkyl, phenyl, aryl and heterocyclyl is optionally substituted with up to three substituents selected from R^{e}.

Preferably R³ is H, cyano, (C₁-C₄)alkyl, (C₂-C₆)alkenyl, N(R^{c})₂, S(O)ₘR^{a}, CF₃ or a ring which is: indolyl, benzofuranyl, chromenyl, tetrahydroisoquinolinyl, pyridinyl, naphthyl, benzodioxolyl, thienyl, thiadiazolyl, cyclopropyl, cyclohexyl, thiazolidinyl, phenyl, benzoyl, isoquinolinyl, cyclopentyl, indolylcarbonyl, bicycloheptyl, pyrazinyl, piperidinyl, napthyridinyl, quinoxalinyl, quinolinyl, pyrazolyl, dihydroisoindolyl, triazolyl, hydrobenzoxazolyl, thiazolyl, dihydrotriazolyl, dihydrobenzodioxinyl, imidazolyl, azepanyl, isoxazolyl, pyrrolyl, furylcarbonyl, cycloheptyl, benzimidazolyl, dihydrobenzofuryl, phenoxyethyl, tetrahydropyranyl, morpholinyl, piperazinyl, triazolopyrimidinyl, pyrrolidinyl, dihydroimidazolyl, oxazolidinyl, benzimidazolylethyl, azetidinyl, azabicycloheptyl, octahydroisoindolyl, benzothiadiazolyl, dihydrobenzoxazinyl, benzothienyl or dihydrobenzoxazolyl; said alkyl, alkenyl or ring being optionally substituted by up to three substituents selected from R^{e}

In an embodiment, R³ is heterocycle; optionally substituted by up to three substituents selected from R^{e}. Preferred optionally substituted heterocycles are indolyl and thienyl. More particularly, R³ is (methyl)(methoxy)indoly or thienyl.

More specifically, R³ is selected from

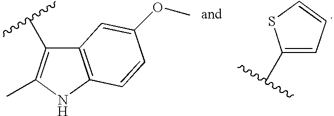

In an embodiment, R^{a} is independently selected from: (C₁-C₆)alkyl, said alkyl is optionally substituted with one or more substituents selected from OH, (C₁-C₆)alkyl, (C₁-C₆)alkoxy, halogen, CO₂H, CN, (O)C═O(C₁-C₆)alkyl, oxo and N(R^{c})₂.

Preferably, R^{a} is H, (C₁-C₆)alkyl, (C═O)O(C₁-C₆)alkyl, phenyl or pyridinyl. More specifically, R^{a} is H, methyl, ethyl, phenyl, pyridin-4-yl or tertbutoxycarbonyl.

In an embodiment, R^{b} is independently selected from: H, OH, Oₐ(C₁-C₆)alkyl, N(R^{c})₂ and phenyl; said alkyl and phenyl is optionally substituted with phenyl and N(R^{g})₂.

In an embodiment, R^{b} is independently selected from: H, OH, Oₐ(C₁-C₆)alkyl, N(R^{c})₂ and phenyl-NH₂; said alkyl is optionally substituted with phenyl and N(R^{g})₂.

Preferably, each R^{b} is independently H, benzyloxy, aminophenyl, OH, methoxy, methyl, ethoxy, butoxy, dimethylamino, benzyl or phenylethyl.

More particularly, each R¹ is independently H, benzyloxy, 2-aminophenyl, OH, methoxy, methyl, ethoxy, tert-butoxy, dimethylamino, benzyl or 2-phenylethyl.

Thus, specific R² groups include OH, benzyloxyamino, (2-aminophenyl)amino, methoxy, hydroxyamino, (methoxy)(methyl)amino, amino, (hydroxy)(methyl)amino, methoxyamino, ethoxyamino, tert-butoxyamino, methylamino, dimethylamino, 2,2-dimethylhydrazino, benzylamino and (2-phenylethyl)amino.

In an embodiment, R^{c} is independently selected from: H, (C═O)ₐO_{b}(C₁-C₆)alkyl-phenyl and (C═O)ₐO_{b}(C₁-C₆)alkyl.

Preferably, R^{c} is H, (C═O)(C₁-C₆)alkyl, (C₁-C₆)alkyl, (C═O)O(C₁-C₆)alkyl-phenyl and (C₁-C₆)alkyl-phenyl. More particularly, R^{c} is H, acetyl, methyl, ethyl, benzyl or benzoxycarbonyl.

In another embodiment, R^{c} is independently selected from: H and (C₁-C₆)alkyl.

More particularly, R^{c} is methyl.

In an embodiment, R^{d} is independently selected from: NO₂, Oₐ-aryl, Oₐ-heterocyclyl, NH(C═O)-aryl, NH(C═O)(C₁-C₆)alkyl, (C═O)N(R^{c})₂, Oₐ-perfluoroalkyl, OₐCF₃, (C═O)ₐ(C₁-C₆)alkyl, NHS(O)ₘ-aryl, NHS(O)ₘ(C₁-C₆)alkyl, N(R^{c})₂, Oₐ(C₁-C₆)alkyl-heterocyclyl, Oₐ(C₁-C₆)alkyl-N(R^{g})₂, S(O)ₘ(C₁-C₆)alkyl, S(O)ₘ-aryl, (C═O)ₐ— aryl, Oₐ(C₁-C₆)alkyl, CN, S(O)ₘN(R^{c})₂, oxo, OH and halo; wherein said alkyl, aryl and heterocyclyl are optionally substituted with R^{f}.

In another embodiment, R^{d} is independently selected from: (C═O)ₐ-phenyl, (C₁-C₆)alkylₐ-heterocyclyl, Oₐ(C₁-C₆)alkyl, oxo, CN, S(O)ₘN(R^{c})₂, OH and halo; wherein said heterocyclyl is selected from:

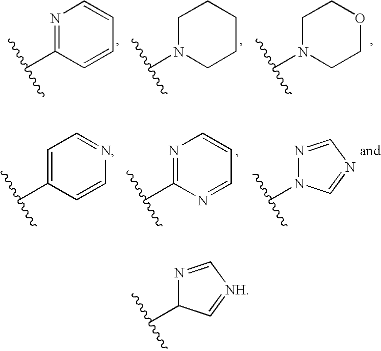

Further R^{d} groups include pyridin-3-yl, (C═O)(C₁-C₆)alkyl, CF₃, pyrrol-1-yl and NH(C═O)(C₁-C₆)alkyl.

In another embodiment, R^{d} is independently selected from: H, CH₃ and (C═O)ₐ-phenyl.

In still another embodiment, R^{d} is phenyl.

Preferably, R^{d} is cyano, halo, oxo, OH, (C₁-C₆)alkyl, O(C₁-C₆)alkyl, (C═O)(C₁-C₆)alkyl, SO₂N(R^{c})₂, NH(C═O)(C₁-C₆)alkyl, CF₃ or a ring which is phenyl, triazolyl, imidazolyl, morpholinyl, pyrimidinyl, pyridinyl, benzoyl, piperidinyl or pyrrolyl; said alkyl or ring optionally substituted by up to three substituents selected from R^{f}.

More particularly, R^{d} is phenyl, triazolyl, methyl, imidazolyl, benzyl, methoxy, morpholinyl, oxo, isopropyl, pyrimidinyl, pyridinylmethyl, fluorine, hydroxy, aminosulfonyl, benzoyl, methoxyphenyl, pyridinyl, piperidinyl, chlorine, cyano, acetyl, trifluoromethyl, pyrrolyl, ethoxy, acetylamino and ethyl.

Specifically, R^{d} is halo or phenyl. More particularly, R^{d} is chlorine or phenyl.

In an embodiment, R^{e} is independently selected from: (C═O)ₐCF₃, oxo, OH, halogen, CN, N(R^{c})₂, NO₂, (C═O)ₐO_{b}(C₁-C₁₀)alkyl, (C═O)ₐO_{b}(C₂-C₁₀)alkenyl, (C═O)ₐO_{b}(C₂-C₁₀)alkynyl, (C═O)ₐO_{b}(C₃-C₈)cycloalkyl, (C═O)ₐO_{b}(C₁-C₆)alkylene-aryl, (C═O)ₐO_{b}-aryl, (C═O)ₐO_{b}(C₁-C₆)alkylene-heterocyclyl, (C═O)ₐO_{b}-heterocyclyl, NH(C═O)ₐ-aryl, (C₁-C₆)alkyl(O)ₐ-phenyl, (C═O)ₐO_{b}(C₁-C₆)alkylene-N(R^{a})₂, N(R^{a})₂, O_{b}(C₁-C₃)perfluoroalkyl (C₁-C₆)alkylene-S(O)ₘR^{a}S(O)ₘR^{a}, C(O)R^{a}, (C₁-C₆)alkylene-CO₂R^{a}, CO₂R^{a}, C(O)H, (C₁-C₆)alkylₐNH(C₁-C₆)alkyl-N(R^{c})₂, C(O)N(R^{a})₂, and S(O)₂N(R^{a})₂.

In another embodiment, R^{e} is independently selected from: (C═O)ₐ—CF₃, oxo, OH, halogen, CN, N(R^{c})₂, S(O)₂(C₁-C₆)alkyl, (C₁-C₆)alkylₐ(C═O)NH(C₁-C₆)alkyl-N(R^{c})₂, O(C₁-C₆)alkyl-N(R^{c})₂, (C═O)ₐO_{b}(C₁-C₁₀)alkyl, (C₁-C₆)alkyl-phenyl, aryl, heterocyclyl and S(O)₂-phenyl.

In yet another embodiment, R^{e} is independently selected from: (C═O)ₐ—CF₃, oxo, OH, halogen, CN, N(R^{c})₂, S(O)₂(C₁-C₆)alkyl, (C₁-C₆)alkylₐ(C═O)NH(C₁-C₆)alkyl-N(R^{c})₂, O(C₁-C₆)alkyl-N(R^{c})₂, (C═O)ₐO_{b}(C₁-C₁₀)alkyl, (C₁-C₆)alkyl-phenyl, aryl, heterocyclyl, S(O)₂-phenyl; wherein said heterocyclyl is selected from:

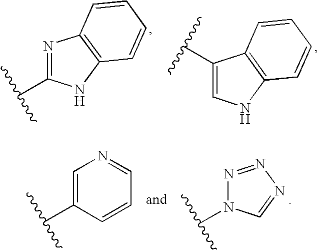

Further R^{e} groups include (C₂-C₁₀)alkenyl, O—CF₃ and pyrrol-1-yl.

Preferably, R^{e} is bromine, chlorine, fluorine, oxo, cyano, methyl, ethyl, isopropyl, trifluoromethyl, acetyl, trifluoroacetyl, methoxy, diethylamino, acetylamino, methylsulfonyl, phenylsulfonyl, [(aminohexyl)amino](oxo)ethyl, [(benzyloxycarbonylamino)hexylamino](oxo)ethyl, (butyloxycarbonylamino)hexoxy, hexenyl, trifluoromethoxy; or a phenyl, benzyl, pyridinyl, tetrazolyl, pyrazolyl or indolyl ring.

More particularly, R^{e} is (C₁-C₆)alkyl or O(C₁-C₆)alkyl. More specifically, R^{e} is methyl or methoxy.

In an embodiment, R^{f} is selected from: phenyl, heterocyclyl and Oₐ(C₁-C₆)alkyl.

Preferably, R^{f} is phenyl, methoxy or pyridinyl.

In another embodiment, R^{f} is selected from: phenyl and Oₐ(C₁-C₆)alkyl.

In an embodiment, R^{g} is independently selected from: H and (C₁-C₆)alkyl.

Preferably R^{g} is H.

In an embodiment of the invention,

R¹ is phenylindolyl, chlorophenyl or quinolinyl; and

R³ is (methoxy)(methyl)indolyl or thienyl.

In another embodiment of the invention,

R¹ is 2-phenyl-1H-indol-3-yl, 4-chlorophenyl or quinolin-3-yl; and

R³ is 5-methoxy-2-methyl-1H-indol-3-yl or 2-thienyl.

In an embodiment R³ is not thienyl.

Another embodiment of the instant invention is a compound as illustrated by formula III:

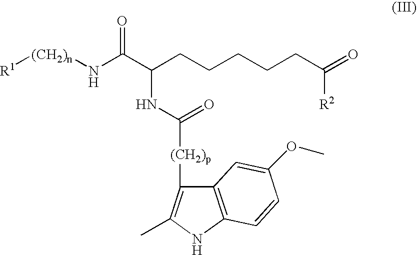

wherein:
all substituents and variables are as defined above:
or a pharmaceutically acceptable salt or stereoisomer thereof.

Another embodiment of the instant invention is a compound as illustrated by formulae IV:

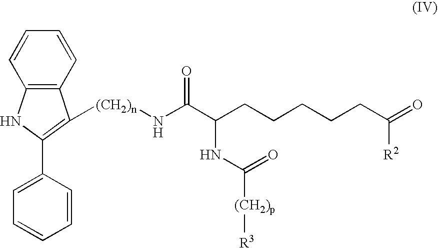

wherein:
all substituents and variables are as defined above
or a pharmaceutically acceptable salt or stereoisomer thereof.

The preferred identities with reference to formula III and IV are as defined previously mutatis mutandis.

Included in the instant invention is the free form of compounds of Formula I, as well as the pharmaceutically acceptable salts and stereoisomers thereof. Some of the specific compounds exemplified herein are the protonated salts of amine compounds. The term “free form” refers to the amine compounds in non-salt form. The encompassed pharmaceutically acceptable salts not only include the salts exemplified for the specific compounds described herein, but also all the typical pharmaceutically acceptable salts of the free form of compounds of Formula I. The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the compounds of this invention include the conventional non-toxic salts of the compounds of this invention as formed by reacting a basic instant compound with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

The compounds of this invention may be prepared by employing reactions as shown in the following schemes, in addition to other standard manipulations that are known in the literature or exemplified in the experimental procedures. The illustrative schemes below, therefore, are not limited by the compounds listed or by any particular substituents employed for illustrative purposes. Substituent numbering as shown in the schemes does not necessarily correlate to that used in the claims and often, for clarity, a single substituent is shown attached to the compound where multiple substituents are allowed under the definitions of Formula I hereinabove

### Reaction Schemes

As shown in Scheme A, HDAC inhibitors can readily be prepared, using the general chemistry outlined below, from protected amino α, ω-diacids. This chemistry can be performed on racemic material, S-amino acids as illustrated or the corresponding R-amino acid. These amino α, ω-diacids can be prepared by those skilled in the art using standard chemistry, such as described in Williams, R. M. Synthesis of Optically Active α-Amino Acids, Pergamon Press, 1989. The key protected amino acid can be O-deprotected, coupled, and then N-deprotected and coupled, to yield after final deprotection of the ω-acid and coupling with an amine, hydroxylamine or hydrazine derivative the desired inhibitors. The ω-carboxylic acids can also be used as inhibitors in there own right, or alternatively converted to ester derivatives. Alternatively, depending on protecting groups, these steps can be reversed, firstly coupling the N-terminus and then the C-terminus prior to final functionalisation of the ω-acid. Suitable methodology is described in Bodanszky, M. Peptide Chemistry, A Practical Textbook 2nd Edition, Springer-Verlag, 1993 and Jones, J. Amino Acid and Peptide Synthesis, Oxford University Press, 1992. Coupling procedures, methods for coupling carboxylic acids (and acid derivatives) with amines to form carboxamides are well known in the art, suitable methods are described, for example, in March, J. Advanced Organic Chemistry, 3rd edition, John Wiley & Sons, 1985, pp. 370-376. In some cases further synthetic manipulation on the complete molecule can lead to other analogues.

The required differentially protected amino α, ω-diacids can readily be prepared, one approach is illustrated in Scheme B whereby alkylation of a lithiated Schollkopf derivative with a suitably functionalised alkyl iodide (for instance bearing a base stable tert-butyl ester) gives after mild acid hydrolysis a chiral α-amino diester (see U. Schollkopf et al. Synthesis 1982, 866). Manipulation of this intermediate first by coupling the amino terminus and then basic hydrolysis of the methyl ester and subsequent coupling yields a protected carboxylic acid. Removal of the protecting group, for instance with TFA, liberateds the free acid which can be coupled to yield the desired HDAC inhibitors.

As shown in Scheme C, the compounds can be further manipulated, for instance, if the reacting partner contains a suitable functional group this can be reacted to yield other compounds. For example an acid can then be esterified esterified to give compounds bearing a terminal ester group (e.g. with TMS-diazomethane). The carboxylic acid can also be coupled with amines to give amides, or with hydrazine derivatives to yield hydrazides, or with hydroxylamines to form the corresponding hydroxamates.

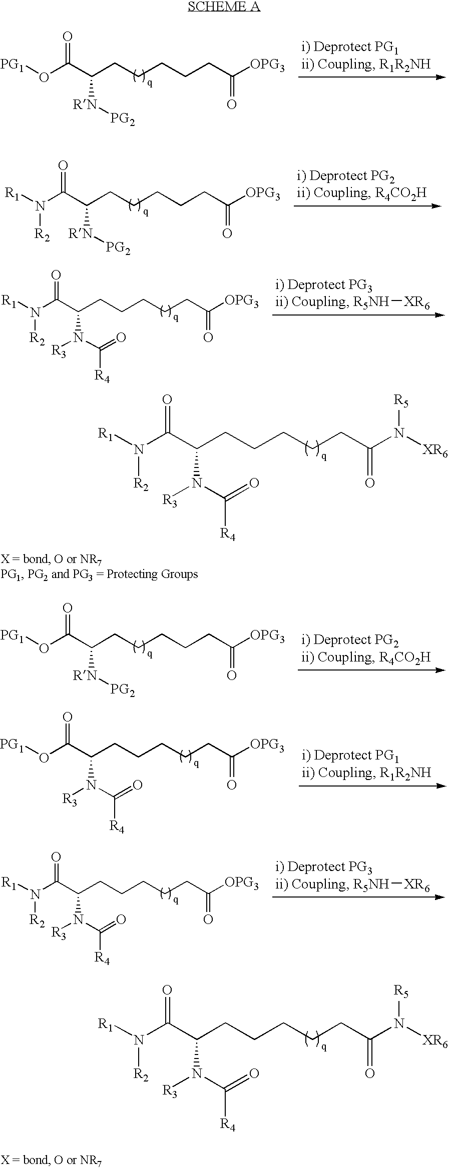

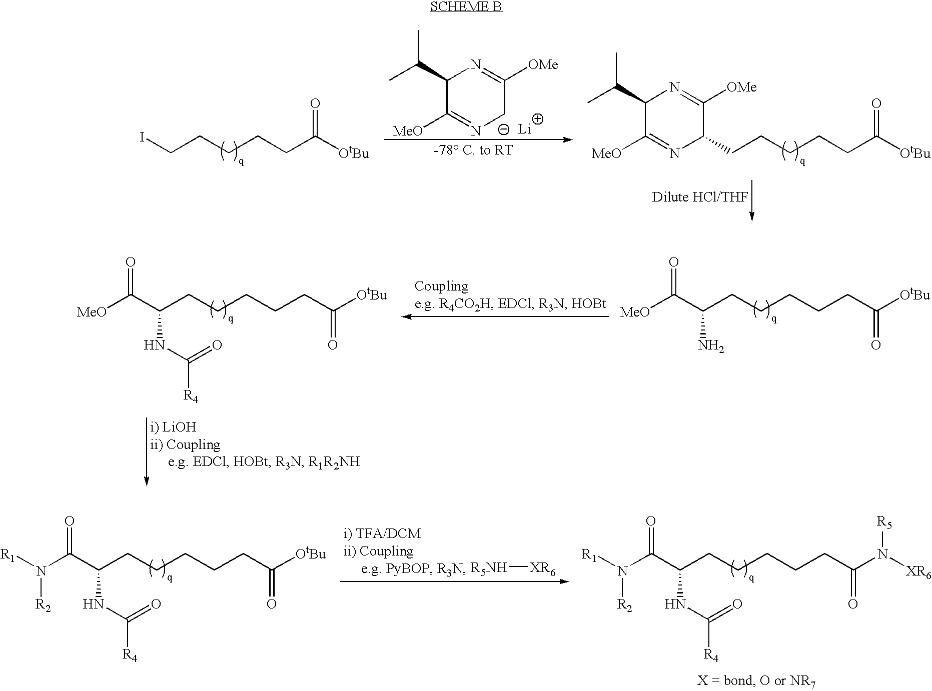

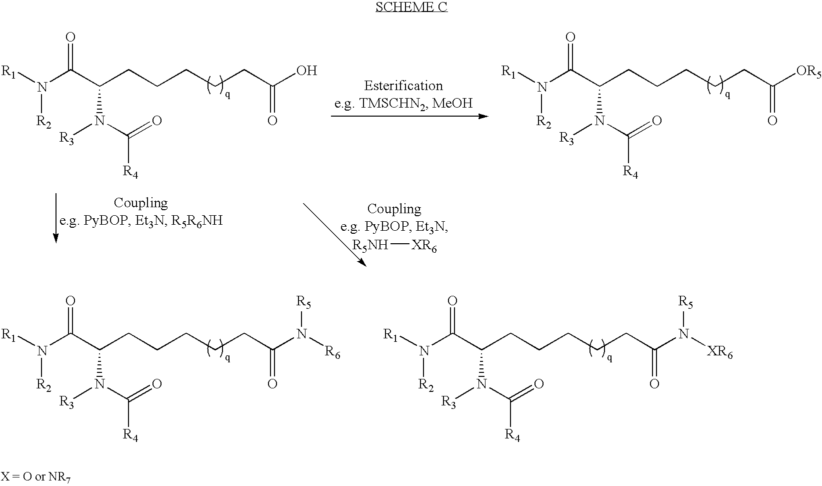

### UTILITY

The compounds of the invention can be used in a method of treatment of the human or animal body by therapy.

The compounds of the invention find use in a variety of applications. The compounds of the invention are histone deacetylase (HDAC) inhibitors useful in the treatment of cancer among other diseases. HDACs catalyse the removal of acetyl groups from lysine residues on proteins, including histones and HDAC inhibitors show diverse biological functions including affecting gene expression, cell differentiation, cell cycle progression, growth arrest, and/or apoptosis. See J. Med. Chem. 2003, 46:5097 and Curr. Med. Chem. 2003, 10:2343.

The compounds of the invention are used to treat cellular proliferation diseases. Disease states which can be treated by the methods and compositions provided herein include, but are not limited to, cancer (further discussed below), neurodegenerative diseases, schizophrenia and stroke

The compounds, compositions and methods provided herein are particularly deemed useful for the treatment of cancer including solid tumors such as skin, breast, brain, cervical carcinomas, testicular carcinomas, etc. In particular, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

The compounds of the invention are also useful in preparing a medicament that is useful in treating the cellular proliferation diseases above, in particular cancer.

The present invention also provides a method for the treatment of cellular proliferation diseases, which method comprises administration to a patient in need thereof of an effective amount of a compound of this invention.

The compounds of the instant invention may also be useful in the treatment or prevention of neurodegenerative diseases, including, but not limited to, polyglutamine-expansion-related neurodegeneration, Huntington's disease, Kennedy's disease, spinocerebellar ataxia, dentatorubral-pallidoluysian atrophy (DRPLA), protein-aggregation-related neurodegeneration, Machado-Joseph's disease, Alzheimer's disease, Parkinson's disease, amyotrophic lateral sclerosis, spongiform encephalopathy, a prion-related disease and multiple sclerosis (MS). See WO 02/090534 and WO 03/083067.

The compounds of the invention are also useful in preparing a medicament that is useful in treating or preventing neurodegenerative diseases.

The present invention also provides a method for treating or preventing neurodegenerative diseases, which method comprises administration to a patient in need thereof of an effective amount of a compound of this invention.

The compounds of the invention may also be useful in the treatment or prevention of schizophrenia. See WO 02/090534.

The compounds of the invention are also useful in preparing a medicament that is useful in treating or preventing schizophrenia.

The present invention also provides a method for treating or preventing schizophrenia, which method comprises administration to a patient in need thereof of an effective amount of a compound of this invention.

The compounds of the invention may also be useful in the treatment or prevention of inflammatory diseases, including, but not limited to stroke, rheumatoid arthritis, lupus erythematosus, ulcerative colitis and traumatic brain injuries. See Leoni et al., PNAS, 99(5):2995-3000 (2002), Suuronen et al., J. Neurochem. 87:407-416 (2003) and Drug Discovery Today, 10:197-204 (2005).

The compounds of the invention are also useful in preparing a medicament that is useful in treating or preventing inflammatory diseases such as stroke.

The present invention also provides a method for treating or preventing inflammatory diseases, which method comprises administration to a patient in need thereof of an effective amount of a compound of this invention.

The compounds of the invention may also be useful in the treatment or prevention of mental retardation, in particular “X chromosome-linked mental retardation” and “Rubinstein-Taybi syndrome”.

The compounds of the invention are also useful in preparing a medicament that is useful in treating or preventing mental retardation.

The present invention also provides a method for treating or preventing mental retardation, which method comprises administration to a patient in need thereof of an effective amount of a compound of this invention.

The compounds of the present invention are also useful in the inhibition of smooth muscle cell proliferation and/or migration and are thus useful in the prevention and/or treatment of restenosis, for example after angioplasty and/or stent implantation.

The compounds of the invention are also useful in preparing a medicament that is useful in treating or preventing restenosis.

The present invention also provides a method for treating or prevention restenosis, which method comprises administration to a patient in need thereof of an effective amount of a compound of this invention.

In one embodiment, smooth muscle cell proliferation and/or migration is inhibited and restenosis is prevented and/or treated by providing a stent device having one or more of the compounds of the instant invention in or on the stent device, e.g. coated onto the stent device. The stent device is designed to controllably release the compounds of the invention, thereby inhibiting smooth miscle cell proliferation and/or migration and preventing and/or treating restenosis.

Stenosis and restenosis are conditions associated with a narrowing of blood vessels. Stenosis of blood vessels generally occurs gradually over time. Restenosis, in contrast, relates to a narrowing of blood vessels following an endovascular procedure, such as balloon angioplasty and/or stent implantation, or a vascular injury.

Balloon angioplasty is typically performed to open a stenotic blood vessel; stenting is usually performed to maintain the patency of a blood vessel after, or in combination with, balloon angioplasty. A stenotic blood vessel is opened with balloon angioplasty by navigating a balloon-tipped catheter to the site of stenosis, and expanding the balloon tip effectively to dilate the occluded blood vessel. In an effort to maintain the patency of the dilated blood vessel, a stent may be implanted in the blood vessel to provide intravascular support to the opened section of the blood vessel, thereby limiting the extent to which the blood vessel will return to its occluded state after release of the balloon catheter. Restenosis is typically caused by trauma inflicted during angioplasty, effected by, for example, ballon dilation, atherectomy or laser ablation treatment of the artery. For these procedures, restenosis occurs at a rate of about 30% to about 60% depending on the vessel location, lesion length and a number of other variables. This reduces the overall success of the relatively non-invasive balloon angioplasty and stenting procedures.

Restenosis is attributed to many factors, including proliferation of smooth muscle cells (SMC). SMC proliferation is triggered by the initial mechanical injury to the intima that is sustained at the time of balloon angioplasty and stent implantation. The process is characterized by early platelet activation and thrombus formation, followed by SMC recruitment and migration, and, finally, cellular proliferation and extracellular matrix accumulation. Damaged endothelial cells, SMCs, platelets, and macrophages secrete cytokines and growth factors which promote restenosis. SMC proliferation represents the final common pathway leading to neointimal hyperplasia. Therefore, anti-proliferative therapies aimed at inhibiting specific regulatory events in the cell cycle may constitute the most reasonable approach to restenosis after angioplasty.

The compounds of the invention may also be used as immunosuppressants or immunomodulators and can accordingly be used in the treatment or prevention of immune response or immune-mediated responses and diseases such as systemic lupus erythematosus (SLE) and acute or chronic transplant rejection in a recipient of an organ, tissue or cell transplant, (see WO 05/013958).

Examples of autoimmune diseases for which the compounds of the invention may be employed include autoimmune hematological disorders (including hemolytic anaemia, aplastic anaemia, pure red cell anaemia and idiopathic thrombocytopenia), systemic lupus erythematosus, thyroiditis, Hashimoto's thyroiditis, polychondritis, sclerodoma, Wegener granulamatosis, dermatomyositis, chronic active hepatitis, myasthenia gravis, psoriasis, atopic dermatitis, vasculitis, Steven-Johnson syndrome, idiopathic sprue, autoimmune inflammatory bowel disease (including ulcerative colitis and Crohn's disease) endocrine opthalmopathy, Graves disease, sarcoidosis, multiple sclerosis, primary billiary cirrhosis, juvenile diabetes (diabetes mellitus type I), diabetes type II and the disorders associated therewith, uveitis (anterior and posterior), keratoconjunctivitis sicca and vernal keratoconjunctivitis, interstitial lung fibrosis, psoriatic arthritis, glomerulonephritis (with and without nephrotic syndrome, including idiopathic nephrotic syndrome or minimal change nephropathy), juvenile dermatomyositisinfectious, auto-antibody mediated diseases, aplastic anemia, Evan's syndrome, autoimmune hemolytic anemia, infectious diseases causing aberrant immune response and/or activation, such as traumatic or pathogen induced immune disregulation, including for example, that which are caused by hepatitis B and C infections, staphylococcus aureus infection, viral encephalitis, sepsis, parasitic diseases wherein damage is induced by inflammatory response (e.g. leprosy); and circulatory diseases, such as arteriosclerosis, atherosclerosis, polyarteritis nodosa and myocarditis.

The compounds of the invention are also useful in preparing a medicament that is useful for the treatment or prevention of immune disorders.

The present invention also provides a method for treating or preventing immune disorders, which method comprises administration to a patent in need thereof of an effective amount of a compound of this invention.

The compounds of the invention may also be useful in the treatment or prevention of other diseases such as diabetes, cardiovascular disorders and asthma.

The compounds of this invention may be administered to mammals, preferably humans, either alone or in combination with pharmaceutically acceptable carriers, excipients or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, sex and response of the individual patient, as well as the severity of the patient's symptoms.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

The instant compounds are also useful in combination with known therapeutic agents and anti-cancer agents. Thus, this invention provides combinations of compounds of formula (I) and known therapeutic agents and/or anti-cancer agents for simultaneous, separate or sequential administration. For example, instant compounds are useful in combination with known anti-cancer agents. Combinations of the presently disclosed compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: other HDAC inhibitors, estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The instant compounds are particularly useful when co-administered with radiation therapy.

In an embodiment, the instant compounds are also useful in combination with known anti-cancer agents including the following: other HDAC inhibitors, estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

Examples of “other HDAC inhibitors” include suberoylanilide hydroxamic acid (SAHA), LAQ824, LBH589, PXD101, MS275, FK228, valproic acid, butyric acid and CI-994.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunorubicin (see WO 00/50032).

An example of a hypoxia activatable compound is tirapazamine.

Examples of proteasome inhibitors include but are not limited to lactacystin, bortezomib, epoxomicin and peptide aldehydes such as MG 132, MG 115 and PSI.

Examples of microtubule inhibitors/microtubule-stabilising agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768, WO 01/98278, WO 03/050,064, WO 03/050,122, WO 03/049,527, WO 03/049,679, WO 03/049,678 and WO 03/39460 and pending PCT Appl. Nos. US03/06403 (filed Mar. 4, 2003), US03/15861 (filed May 19, 2003), US03/15810 (filed May 19, 2003), US03/18482 (filed Jun. 12, 2003) and US03/18694 (filed Jun. 12, 2003). In an embodiment inhibitors of mitotic kinesins include, but are not limited to inhibitors of KSP, inhibitors of MKLP1, inhibitors of CENP-E, inhibitors of MCAK, inhibitors of Kif14, inhibitors of Mphosphl and inhibitors of Rab6-KIFL.

“Inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,1′-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and U.S. Ser. No. 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Inhibitors of cell proliferation and survival signaling pathway” refer to pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in (WO 03/086404, WO 03/086403, WO 03/086394, WO 03/086279, WO 02/083675, WO 02/083139, WO 02/083140 and WO 02/083138), inhibitors of Raf kinase (for example BAY-43-9006), inhibitors of MEK (for example CI-1040 and PD-098059) and inhibitors of mTOR (for example Wyeth CCI-779 and Ariad AP23573). Such agents include small molecule inhibitor compounds and antibody antagonists.

“Apoptosis inducing agents” include activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272, and U.S. Pat. No. 5,932,598, all of which are hereby incorporated by reference.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to: parecoxib, CELEBREX® and BEXTRA® or a pharmaceutically acceptable salt thereof.

Other examples of angiogenesis inhibitors include, but are not limited to, endostatin, ukrain, ranpimase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ5 integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malignancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Ophthalmol. Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. No. 60/235,708 and 60/244,697).

Another embodiment of the instant invention is the use of the presently disclosed compounds in combination with anti-viral agents (such as nucleoside analogs including ganciclovir for the treatment of cancer. See WO 98/04290.

Another embodiment of the instant invention is the use of the presently disclosed compounds in combination with gene therapy for the treatment of cancer. For an overview of genetic strategies to treating cancer see Hall et al (Am J Hum Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8): 1105-13), and interferon gamma (J Immunol 2000; 164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In an embodiment, an anti-emesis agent selected from a neurokinin-1 receptor antagonist, a 5HT3 receptor antagonist and a corticosteroid is administered as an adjuvant for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, isoprinosine and Zadaxin.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

Thus, the scope of the instant invention encompasses the use of the instantly claimed compounds in combination with a second compound selected from: other HDAC inhibitors, an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an anti-viral agent, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an agent that interfers with a cell cycle checkpoint, an apoptosis inducing agent, and a bisphosphonate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In an embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Also included in the scope of the claims is a method of treating cancer that comprises administering a therapeutically effective amount of a compound of Formula I in combination with radiation therapy and/or in combination with a compound selected from: other HDAC inhibitors, an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an anti-viral agent, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an agent that interfers with a cell cycle checkpoint, an apoptosis inducing agent, and a bisphosphonate.

And yet another embodiment of the invention is a method of treating cancer that comprises administering a therapeutically effective amount of a compound of Formula I in combination with paclitaxel or trastuzumab.

The invention further encompasses a method of treating or preventing cancer that comprises administering a therapeutically effective amount of a compound of Formula I in combination with a COX-2 inhibitor.

The instant invention also includes a pharmaceutical composition useful for treating or preventing cancer that comprises a therapeutically effective amount of a compound of Formula I and a compound selected from: other HDAC inhibitors, an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an anti-viral agent, an inhibitor of cell proliferation and survival signaling, an agent that interfers with a cell cycle checkpoint, an apoptosis inducing agent, and a bisphosphonate.

These and other aspects of the invention will be apparent from the teachings contained herein.

All patents, publications and pending patent applications identified are hereby incorporated by reference.

Abbreviations used in the description of the chemistry and in the Examples that follow are: AcOH (acetic acid); BuLi (n-butyl lithium); BSA (bovine serum albumin); DCE (1,2-dichloroethane); DIBAL-H (diisobutylaluminum hydride); DIEA (diisopropylethylamine); DCM (dichloromethane); DME (ethylene glycol dimethyl ether); DMEM (Dulbecco's Modified Eagle Medium); DMF (dimethylformamide); DMSO (dimethyl sulfoxide); DTT (dithiothreitol); EDCI (N-(3-dimethylaminopropyl)-N′-ethylcarbodiimide.HCl); EDTA (ethylenediaminetetraacetic acid); EGTA (Ethyleneglycotetraacetic acid); em (emission); Eq. (equivalent); ES (electrospray); EtOAc (ethyl acetate); ex (exitation); FACS (fluorescence activated cell sorting); FITC (Fluorescein isothiocyanate); Hepes ((N-(2-Hydroxyethyl)piperazine)-N′-(2-ethanesulfonic acid)); HOBt (1-hydroxybenzotriazole); HPLC (high performance liquid chromatography); IPTG (Isopropyl-beta-D-thiogalactopyranoside); KHMDS (potassium hexamethyldisilazide); LEP (Lysyl End Peptidase); LDA (lithium diisopropylamide); LHMDS (lithium hexamethyldisilazide); Lys C (Lysyl C endoprotease); mCPBA (m-chloroperoxybenzoic acid); MeOH (methanol); MS (mass spectrometry); NaHMDS (sodium bistrimethylsilylamide); NMR (nuclear magnetic resonance); NP40 (Nonidet P40); PBS (Phosphate buffered saline); PMSF (phenylmethylsulphonyl fluoride); PTSA (p-Toluenesulphonic acid); PyBop (1H-1,2,3-benzotriazol-1-yloxy)(tripyrrolidin-1-yl)phosphonium hexafluorophosphate); RT (room temperature); SCX (Varian or Isolute cation exchange resin); SiO₂ (silica gel); SPA (Scintillation Proximity Assay); TBAI (tetra-n-butylammonium iodide); TEA (triethyl amine); THF (tetrahydrofuran); TFA (trifluoroacteic acid); TMSCN (trimethylsilylcyanide); Tris (Tris Hydroxymethylaminoethane); Trisyl (2,4,6-triisopropylbenzene sulphonyl); TSA (Trichostatin A); and TsCl (p-toluenesulfonyl chloride).

### EXAMPLE 1

### See Compound 20

### (2S)-N⁸-Methoxy-N′-[2-(2-phenyl-1H-indol-3-yl)ethyl]-2-[(2-thienylcarbonyl)amino]octanediamide (A7)

### tert-Butyl 6-[(2S,5R)-5-isopropyl-3,6-dimethoxy-2,5-dihydropyrazin-2-yl]hexanoate (E1)

To a stirred solution of (2R)-2-isopropyl-3,6-dimethoxy-2,5-dihydropyrazine (1.0 eq.) in THF at −78° C., a solution of BuLi (1.6 N in hexanes, 1.0 eq.) was added and stirring was continued for 15 min. A precooled solution of tert-butyl 6-iodohexanoate (1.5 eq.) in THF was added and stirring was continued at −78° C. for 8 hours. The reaction mixture was allowed to warm to RT overnight. The reaction was quenched by the addition of aqueous NH₄Cl solution and the mixture extracted with EtOAc. The organic layer was washed with brine, dried (Na₂SO₄) and concentrated under reduced pressure. The crude was purified by column chromatography, eluting with 4% EtOAc/Petroleum ether to afford (E1). ¹H NMR (300 MHz, CDCl₃) δ 4.05-3.95 (1H, m), 3.93 (1H, t, J=3.3 Hz), 3.69 (3H, s), 3.68 (3H, s), 2.35-2.14 (3H, m), 1.85-1.55 (2H, m), 1.44 (9H, s), 1.39-1.15 (6H, m), 1.58 (3H, d, J=6.7 Hz), 0.69 (3H, d, J=6.7 Hz). MS (ES) C₁₉H₃₄N₂O₄ requires: 354, found: 355 (M+H⁺).

### 8-tert-Butyl 1-methyl (2S)-2-aminooctanedioate (E2)

A solution of tert-butyl 6-[(2S,5R)-5-isopropyl-3,6-dimethoxy-2,5-dihydropyrazin-2-yl]hexanoate (E1) in a mixture of 1N HCl solution (6.0 eq.) and MeCN (0.03M) was stirred at RT for 12 hours. The reaction was neutralised with NaHCO₃ and the MeCN was removed under reduced pressure. The desired material was extracted with EtOAc. The organic layer was washed with brine, dried (Na₂SO₄) and concentrated under reduced pressure. The residue (E2) was directly used in the next step without further purification. MS (ES) C₁₃H₂₅NO₄ requires: 259, found: 260 (M+H⁺).

### 8-tert-Butyl 1-methyl (2S)-2-[(2-thienylcarbonyl)amino]octanedioate (E3)

2-Thienylcarboxylic acid (1.5 eq.), HOBt (1.5 eq.), EDCI (1.5 eq.) were dissolved in DMF and the mixture was stirred for 10 min at RT. A solution containing 8-tert-butyl 1-methyl (2S)-2-aminooctanedioate (E2) (1.0 eq.) in DMF was added and the mixture was stirred overnight. The residue was taken up in EtOAc, washed with NaHCO₃ solution, 1M HCl and brine, dried (Na₂SO₄). The crude was purified by column chromatography, eluting with 30% EtOAc/Petroleum ether to afford (E3). ¹H NMR (300 MHz, CDCl₃) δ 7.55 (1H, d, J=3.7 Hz), 7.49 (1H, d, J=5.1 Hz) 7.10 (1H, dd, J=3.7 Hz, J=5.1 Hz), 6.48 (1H, d, J=7.9 Hz) 4.85-4.72 (1H, m), 3.78 (3H, s), 2.19 (2H, t, J=7.4 Hz), 2.00-1.85 (1H, m), 1.85-1.70 (1H, m), 1.70-1.30 (1SH, m). MS (ES) C₁₈H₂₇NO₅S requires: 369, found: 370 (M+H⁺).

### (2S)-8-tert-Butoxy-8-oxo-2-[(2-thienylcarbonyl)amino]octanoic acid (E4)

To a mixture of 8-tert-butyl 1-methyl (2S)-2-[(2-thienylcarbonyl)amino]octanedioate (E3) (1.0 eq.) in THF and H₂O (2:1) was added LiOH (1.2 eq.) and the resulting mixture was stirred overnight at RT. The reaction was neutralised adding 1M HCl and the organics were extracted with EtOAc, dried (Na₂SO₄) and concentrated under reduced pressure to give the desired acid (E4) which was directly used in the next step. MS (ES) C₁₇H₂₅NO₅S requires: 355, found: 356 (M+H⁺).

### tert-Butyl (7S)-8-oxo-8-{[2-(2-phenyl-1H-indol-3-yl)ethyl]amino}-7-[(2-thienylcarbonyl)amino]octanoate (E5)

(2S)-8-tert-Butoxy-8-oxo-2-[(2-thienylcarbonyl)amino]octanoic acid (E4) (1 eq.) was coupled with 2-(2-phenyl-1H-indol-3-yl)ethanamine [Prepared according to Tetrahedron Letters (1997), 38 (22), 3871-3874 and deprotected with hydrazine hydrate] (1.5 eq.) as described in Example 5 Step 3 and the desired material (E5) was isolated by reverse phase prep. HPLC. ¹H NMR (400 MHz, CDCl₃) δ MS (ES) C₃₃H₃₉N₃O₄S requires: 573, found: 574 (M+H⁺).

### (7S)-8-Oxo-8-{[2-(2-phenyl-1H-indol-3-yl)ethyl]amino}-7-[(2-thienylcarbonyl)amino]octanoic acid (E6)

tert-Butyl (7S)-8-oxo-8-{[2-(2-phenyl-1H-indol-3-yl)ethyl]amino}-7-[(2-thienylcarbonyl)amino]octanoate (E5) (1 eq.) was taken up in DCM/TFA (1:1) and stirred at RT for 3 hours. The reaction mixture was concentrated under reduced pressure, brine was added and the desired material was extracted with EtOAc, dried (Na₂SO₄) and concentrated under reduced pressure to yield the desired acid (E6). which was used directly in the next step. MS (ES) C₂₉H₃₁N₃O₄S requires: 517, found: 518 (M+H⁺).

### (2S)-N⁸-Methoxy-N′-[2-(2-phenyl-1H-indol-3-yl)ethyl]-2-[(2-thienylcarbonyl)amino]octanediamide (E7)

(7S)-8-Oxo-8-{[2-(2-phenyl-1H-indol-3-yl)ethyl]amino}-7-[(2-thienylcarbonyl)amino]octanoic acid (E6) (1 eq.) and O-methylhydroxylamine hydrochloride (1.5 eq.) were coupled in the presence of DIPEA (1 eq.) as described in Example 5 Step 3 and the desired material (E7) was isolated by reverse phase prep. HPLC. ¹H NMR (400 MHz, CDCl₃) δ 11.65 (1H, s), 10.89 (1H, bs), 8.44 (1H, d, J=8.2 Hz), 8.22 (1H, m), 7.93 (1H, d, J=2.8 Hz), 7.75 (1H, d, J=4.2 Hz), 7.71-7.60 (3H, m), 7.49 (2H, t, J=7.4 Hz), 7.36 (2H, t, J=7.4 Hz), 7.20-6.98 (3H, m), 4.43-4.30 (1H, m), 3.54 (3H, s), 3.45-3.28 (2H, m), 2.96 (2H, t, J=7.8 Hz), 1.91 (2H, t, J=7.3 Hz), 1.80-1.10 (8H, m). MS (ES) C₃₀H₃₄N₄O₄S requires: 546, found: 547 (M+H⁺).

### EXAMPLE 2

### See Compound 23

### (2S)-N⁸-Hydroxy-N¹-[2-(2-phenyl-1H-indol-3-yl)ethyl]-2-[(2-thienylcarbonyl) amino]octanediamide (F1)

(7S)-8-Oxo-8-{[2-(2-phenyl-1H-indol-3-yl)ethyl]amino}-7-[(2-thienylcarbonyl)amino]octanoic acid (E6)(1 eq.) and O-tetrahydro-2H-pyran-2-yl)hydroxylamine (7.5 eq.) were coupled in the presence of EDCI (5 eq.) and HOBt (5 eq.) and the desired material was isolated by reverse phase prep. HPLC, during which time the labile THP protecting group was removed. ¹H NMR (300 MHz, d6-MSO) δ 11.17 (1H, br. s), 10.30 (1H, br. s), 8.44 (1H, d, J=7.3 Hz), 8.22 (1H, m), 7.93 (1H, m), 7.75 (1H, d, J=3.5 Hz), 7.71-7.60 (3H, m), 7.49 (2H, t, J=6.7 Hz), 7.36 (2H, t, J=6.7 Hz), 7.20-6.98 (3H, m), 4.43-4.30 (1H, m), 3.45-3.28 (2H, m), 2.96 (2H, t, J=6.3 Hz), 1.91 (2H, t, J=6.3 Hz), 1.8-1.1 (8H, m). MS (ES) C₂₉H₃₂N₄O₄S requires: 532, found: 533 (M+H⁺).

The following compounds can be made according to the Reaction Schemes and Examples 1-2.

| Com- | | | |
|---|---|---|---|
| pound | | Mass | |
| Number | Structure | Seen | Nomenclature |
| 1 | 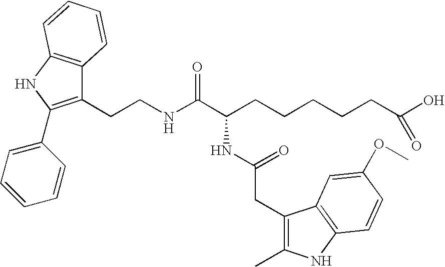 | 609 | (7S)-7-{[(5-Methoxy-2-methyl-1H-indol-3-yl)acetyl]amino}-8-oxo-8-{[2-(2-phenyl-1H-indol-3-yl)ethyl]amino}octanoic acid |
| 2 | 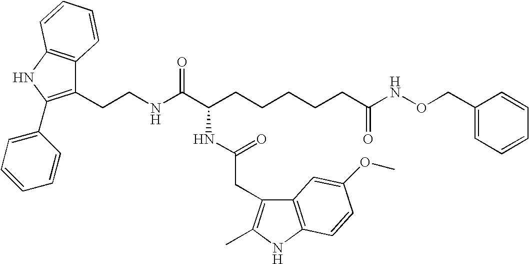 | 714 | (2S)-N⁸-(Benzyloxy)-2-{[(5-methoxy-2-methyl-1H-indol-3-yl)acetyl]amino}-N¹-[2-(2-phenyl-1H-indol-3-yl)ethyl]octanediamide |
| 3 | 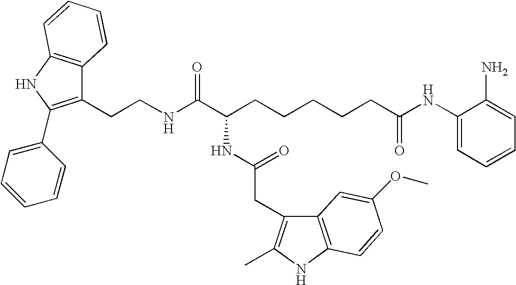 | 699 | (2S)-N⁸-(2-Aminophenyl)-2-{[(5-methoxy-2-methyl-1H-indol-3-yl)acetyl]amino}-N¹-[2-(2-phenyl-1H-indol-3-yl)ethyl]octanediamide |
| 4 | 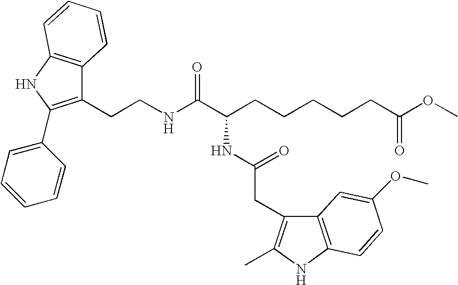 | 623 | Methyl (7S)-7-{[(5-methoxy-2-methyl-1H-indol-3-yl)acetyl]amino}-8-oxo-8-{[2-(2-phenyl-1H-indol-3-yl)ethyl]amino}octanoate |
| 5 | 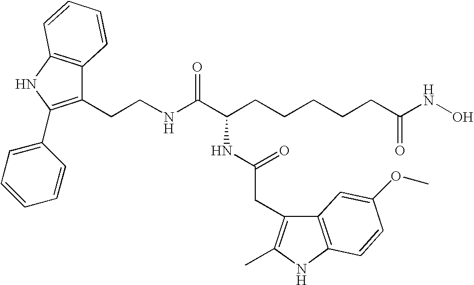 | 624 | (2S)-N⁸-Hydroxy-2-{[(5-methoxy-2-methyl-1H-indol-3-yl)acetyl]amino}-N¹-[2-(2-phenyl-1H-indol-3-yl)ethyl]octanediamide |
| 6 | 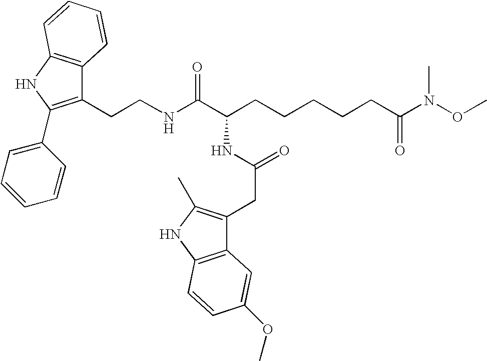 | 652 | (2S)-N⁸-Methoxy-2-{[(5-methoxy-2-methyl-1H-indol-3-yl)acetyl]amino}-N⁸-methyl-N¹-[2-(2-phenyl-1H-indol-3-yl)ethyl]octanediamide |
| 7 | 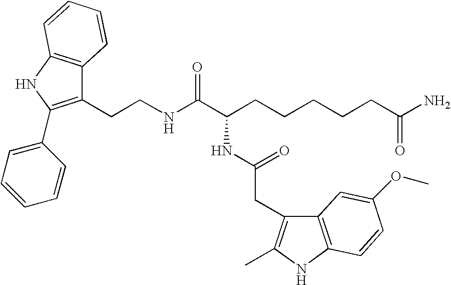 | 608 | (2S)-2-{[(5-Methoxy-2-methyl-1H-indol-3-yl)acetyl]amino}-N¹-[2-(2-phenyl-1H-indol-3-yl)ethyl]octanediamide |
| 8 | 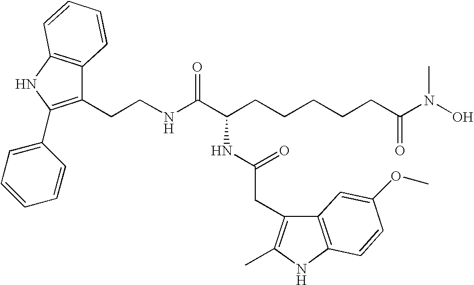 | 638 | (2S)-N⁸-Hydroxy-2-{[(5-methoxy-2-methyl-1H-indol-3-yl)acetyl]amino}-N⁸-methyl-N¹-[2-(2-phenyl-1H-indol-3-yl)ethyl]octanediamide |
| 9 | 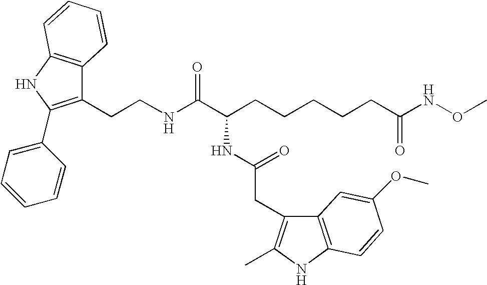 | 638 | (2S)-N⁸-Methoxy-2-{[(5-methoxy-2-methyl-1H-indol-3-yl)acetyl]amino}-N¹-[2-(2-phenyl-1H-indol-3-yl)ethyl]octanediamide |
| 10 | 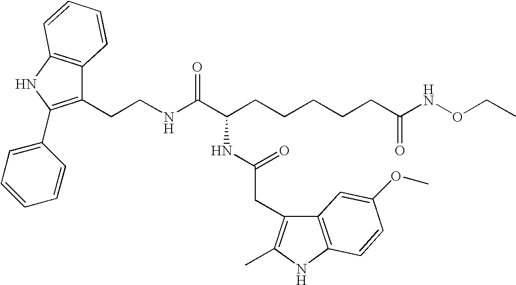 | 652 | (2S)-N⁸-Ethoxy-2-{[(5-methoxy-2-methyl-1H-indol-3-yl)acetyl]amino}-N¹-[2-(2-phenyl-1H-indol-3-yl)ethyl]octanediamide |
| 11 | 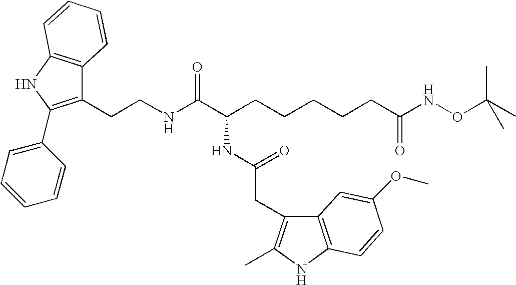 | 681 | (2S)-N⁸-(tert-Butoxy)-2-{[(5-methoxy-2-methyl-1H-indol-3-yl)acetyl]amino}-N¹-[2-(2-phenyl-1H-indol-3-yl)ethyl]octanediamide |
| 12 | 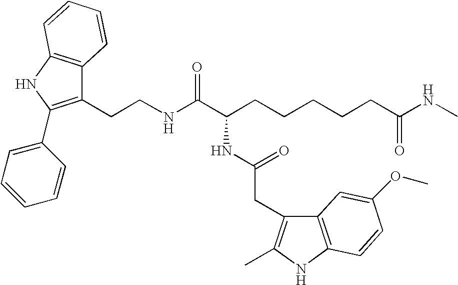 | 622 | (2S)-2-{[(5-Methoxy-2-methyl-1H-indol-3-yl)acetyl]amino}-N⁸-methyl-N¹-[2-(2-phenyl-1H-indol-3-yl)ethyl]octanediamide |
| 13 | 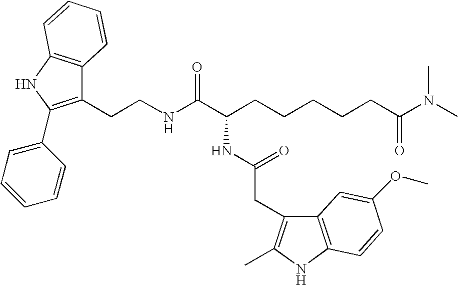 | 636 | (2S)-2-{[(5-Methoxy-2-methyl-1H-indol-3-yl)acetyl]amino}-N⁸,N⁸-dimethyl-N¹-[2-(2-phenyl-1H-indol-3-yl)ethyl]octanediamide |
| 14 | 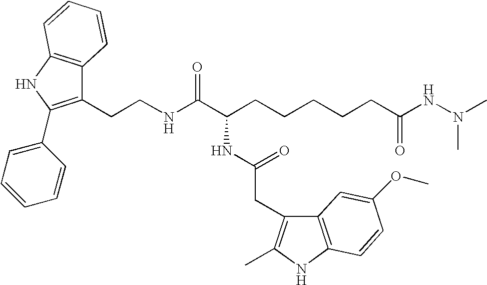 | 651 | (2S)-8-(2,2-Dimethylhydrazino)-2-{[(5-methoxy-2-methyl-1H-indol-3-yl)acetyl]amino}-8-oxo-N-[2-(2-phenyl-1H-indol-3-yl)ethyl]octanamide |
| 15 | 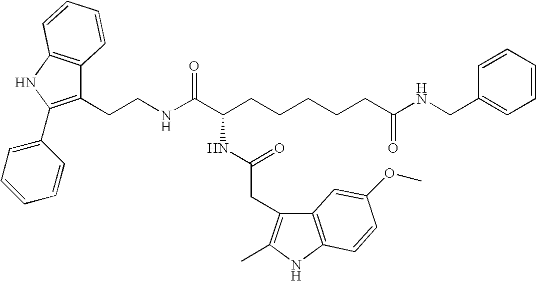 | 698 | (2S)-N⁸-Benzyl-2-{[(5-methoxy-2-methyl-1H-indol-3-yl)acetyl]amino}-N¹-[2-(2-phenyl-1H-indol-3-yl)ethyl]octanediamide |
| 16 | 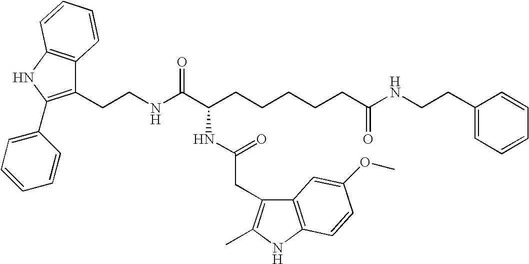 | 712 | (2S)-2-{[(5-Methoxy-2-methyl-1H-indol-3-yl)acetyl]amino}-N⁸-(2-phenylethyl)-N¹-[2-(2-phenyl-1H-indol-3-yl)ethyl]octanediamide |
| 17 | 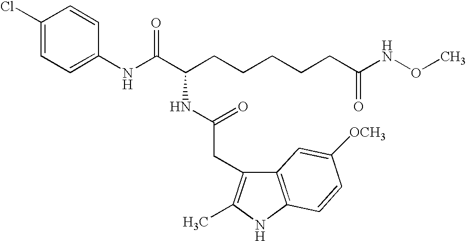 | 529 | (2S)-N¹-(4-Chlorophenyl)-N⁸-methoxy-2-{[(5-methoxy-2-methyl-1H-indol-3-yl)acetyl]amino}octanediamide |
| 18 | 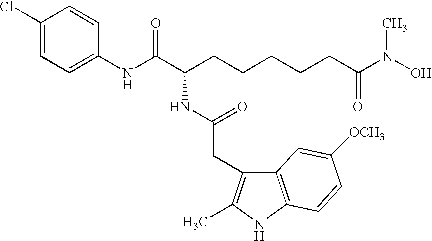 | 529 | (2S)-N¹-(4-Chlorophenyl)-N⁸-hydroxy-2-{[(5-methoxy-2-methyl-1H-indol-3-yl)acetyl]amino}-N⁸-methyloctanediamide |
| 19 | 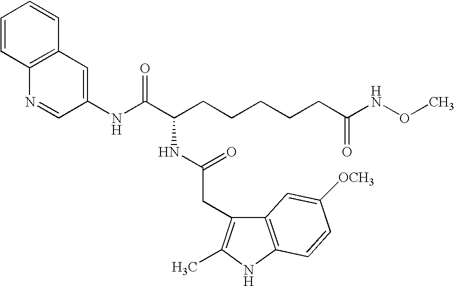 | 546 | (2S)-N⁸-Methoxy-2-{[(5-methoxy-2-methyl-1H-indol-3-yl)acetyl]amino}-N¹-quinolin-3-yloctanediamide |
| 20 | 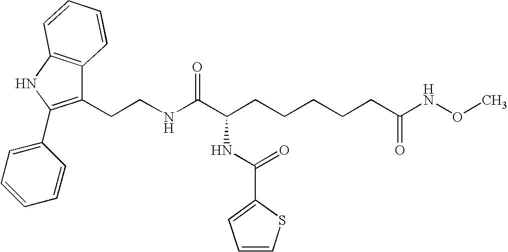 | 547 | (2S)-N⁸-Methoxy-N¹-[2-(2-phenyl-1H-indol-3-yl)ethyl]-2-[(2-thienylcarbonyl)amino]octanediamide |
| 21 | 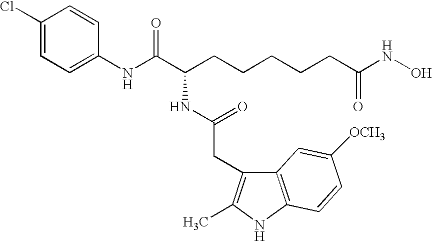 | 515 | (2S)-N¹-(4-Chlorophenyl)-N⁸-hydroxy-2-{[(5-methoxy-2-methyl-1H-indol-3-yl)acetyl]amino}octanediamide |
| 22 | 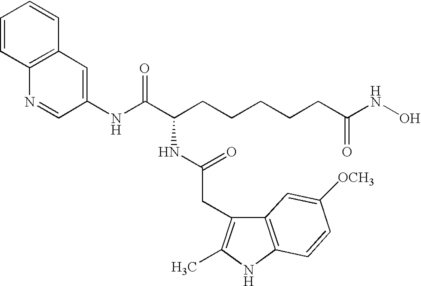 | 532 | (2S)-N⁸-Hydroxy-2-{[(5-methoxy-2-methyl-1H-indol-3-yl)acetyl]amino}-N¹-quinolin-3-yloctanediamide |
| 23 | 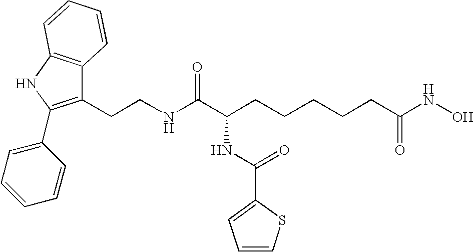 | 533 | (2S)-N⁸-Hydroxy-N¹-[2-(2-phenyl-1H-indol-3-yl)ethyl]-2-[(2-thienylcarbonyl)amino]octanediamide |
| 24 | 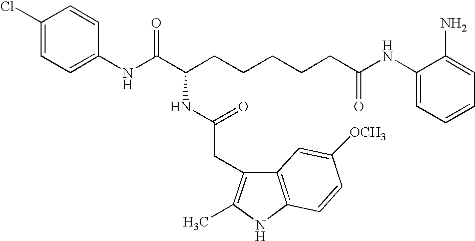 | 590 | (2S)-N⁸-(2-Aminophenyl)-N¹-(4-chlorophenyl)-2-{[(5-methoxy-2-methyl-1H-indol-3-yl)acetyl]amino}octanediamide |
| 25 | 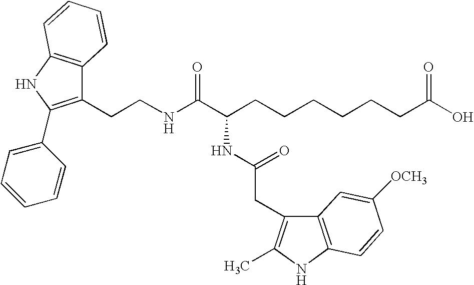 | 623 | (8S)-8-{[(5-Methoxy-2-methyl-1H-indol-3-yl)acetyl]amino}-9-oxo-9-{[2-(2-phenyl-1H-indol-3-yl)ethyl]amino}nonanoic acid |

### Assays

The compounds of the instant invention described in the Examples and shown in Table 1 were tested by the assays described below and were found to have HDAC inhibitory activity (IC₅₀ of <30 μM). Other assays are known in the literature and could be readily performed by those of skill in the art.

### HDAC Assay 1

Prepare 2.5 μl of compound or DMSO (20×) in 96 well microplate Packard Optiplate. To each well add 37.5 μl of Mix A, perform a 30 min. incubation at room temperature while shaking, then add 10 μl of Mix B, perform 3.5 hours incubation at room temperature while shaking, then add 10 μl of STOP Mix, incubate for 30 min. at room temperature and then read in FLUOSTAR ex355 nm em460/40 nm.

The final assay conditions contain: Hepes (pH 7.4, 50mM), Glycerol (10%), BSA (0.1 mg/ml), Triton X100 (0.01%), Fluorogenic peptide IRBM91 (Boc-Ala-Ala-Lys[ε-Ac]-AMC; 20 uM), HeLa S3 extract from nuclei (20 μg/ml) or HDAC1 (1 nM), Lysyl End Peptidase (LEP; 0.25 mAu/ml) or Lysyl C endoprotease (LysC; 4.8mU/ml) and Trichostatin A (1 μM).

The final assay volume is 50 μl.

Mix A contains: Buffer A 1× (37.5 μl), HeLa-S3 extract from nuclei (20 μg/ml; considering 50 μl/well) or HDAC1 (1 nM; considering 50 μl/well).

Mix B contains: Buffer A 1× (10 μl) and Pep IRBM91 (20 μM; considering 50 μl/well).

STOP Mix contains: Buffer A 1× (10 μl), LEP or Lys C (0.25 mAu/ml) or 4.8mU/ml; considering 60 μl final volume) and Trichostatin A (1M; considering 60 μl final volume).

Buffer A 1× contains: Hepes (pH 7.4; 50 mM), Glycerol (10%), BSA (0.1 mg/ml) and Triton X100 (0.01%).

### HDAC Assay 2

Prepare 2.5 μl of compound or DMSO (20×) in 96 well microplate Packard Optiplate.

To each well add 37.5 μl of Mix A, then add 10 μl Mix B, incubate for 3.5 hours at room temperature while shaking, then add 25 μl SPA-Streptavidin beads (in buffer A 1×) and finally read in a Packard TOP COUNT.

The final assay conditions contain: Hepes (pH 7.4, 50 mM), Glycerol (10%), BSA (0.1 mg/nl), Triton X100 (0.01%), 3H Biotin-PEP439 (Biotin-G-A-[acetyl-3H]K—R—H—R-[acetyl-3H]K—V—NH₂, SPA-streptavidin beads (2 mg/ml) and HeLa S3 extract (40 μg/ml).

The final assay volume is 50 μl.

Mix A contains: Buffer A 2× (25 μl), HeLa-S3 extract (40 μg/ml) and H₂O (to 37.5 μl).

Mix B contains: Buffer A 2× (5 μl), Pep 439 (50 nM; considering 50 μl final volume) and H₂O (to 10 μl).

Buffer A 2× contains: Hepes (pH 7.4; 100mM), Glycerol (20%), BSA (0.2 mg/ml) and Triton X100 (0.02%).

Protocol for Nuclei Extraction from HeLa Cells (Adherent or in Suspension).

For a protocol on Nuclei extraction from HeLa S3 cells (adherent or in suspension) refer to Nare et al. 1999 Anal. Biochem., 267: 390-396.

Nuclei preparation for adherent HeLa S3 cells (0.5-1×109 cells) is as follows: wash cells twice with 1×PBS, scrape cells into 1×PBS, wash plates with 1×PBS, pool and spin cells at 800×g 10 minutes at 4° C., wash cell pellets with 1×PBS (count cells), spin cells at 800×g 10 minutes at 4° C., freeze cell pellets in liquid nitrogen and store −80° C.

Nuclei preparation for HeLa S3 cells in suspension (0.5-1×109 cells) is as follows: collect cells by centrifugation at 800×g 10 minutes at 4° C., wash cell pellets with 1×PBS, spin cells at 800×g 10 minutes at 4° C., repeat wash step twice (count cells), freeze cell pellet in liquid nitrogen and store at −80° C.

Resuspend cell pellets in lysis buffer (5 ml/1×108 cells; buffer contains: 0.25M sucrose, 0.45% NP40, 10 mM Tris-HCl (7.5), 10 mM NaCl, 5 mM MgCl₂, 0.1M EGTA, 0.5 mM PMSF, COMPLETE protease inhibitor mix), vortex 10 sec and leave on ice for 15 minutes, spin through cushion (25 ml of lysate/5 ml cushion; cushion contains: 30% sucrose, 10 mM Tris-HCl (7.5), 10mM NaCl, 3 mM MgCl₂), spin through cushion at 1,300×g 10 minutes at 4° C., remove super/cushion, resuspend in lysis buffer as above and re-spin through cushion as above, remove super/cushion.

For nuclear extraction, resuspend nuclear pellets in nuclei extraction buffer (13.5 ml/5 ml nuclear pellet; nuclei extraction buffer contains: 50 mM Hepes pH 7.4, (for use in HDAC ASSAY 2 also include 0.5 mM PMSF and COMPLETE protease inhibitor mix), sonicate into suspension on ice (1 min, output control between 4 and 5), leave on ice 30 min., centrifuge 100,000×g for 1 hr at 4° C., keep super on ice, repeat sonication/ice/centrifuge steps two more times, pool three supernatants and dialyze in 50 mM Hepes pH 7.4/10% glycerol and Snap-freeze suitable aliquots in liquid nitrogen and store −80° C.

### Extraction and Purification Protocol for Flag-Tagged HDAC1 Expressed in HeLa Cells

HeLa cells transiently transfected with pCDNA3-HDAC1-FLAG are grown to 80% confluence on 10 cm culture dishes in DMEM, 10% Fetal bovine serum supplemented with antibiotics and glutamine. Cells are washed with 10 ml cold PBS and scraped into 2 ml of PBS. Cells are centrifuged for 5 minutes at 800×g at 4° C., washed with 30 ml PBS and resuspended in 10 ml PBS, counted, re-centrifuged and frozen at −80° C.

The frozen cell pellet is re-suspended in 1 ml of hypotonic lysis buffer (LB: 20 mM Hepes pH7.9, 0.25 mM EDTA, 10% glycerol) containing COMPLETE protease inhibitor and incubated on ice for 15 minutes, followed by homogenization on a 2-rnl DounceB homogenizer (25 strokes). 150 mM KCl and 0.5% NP-40 are added to the homogenate and the solution is sonicated twice for 30 seconds (output5/6, duty cycle 90) and incubated for 1 hour at 4° C. After a 30 minutes centrifugation at 12000 rpm and 4° C. the supernatant (soluble extract) is collected and protein concentration is determined using the BIORAD assay.

Anti-FLAG M2 affinity resin (Sigma) is washed three times with TBS and twice with LB. 10 μl of the LB-washed resin/mg of protein (2-3 ug of Flagged-HDAC1) are added to the soluble extract (1 mL) and incubated overnight at 4° C. with gentle mixing. The resin is then collected by centrifugation, washed once with LB, twice with LB+0.1% NP40 and twice with elution buffer (50 mM Hepes pH 7.4, 5% glycerol, 100 mM KCl, 0.01% Triton X-100).

The affinity-purified HDAC is eluted from the resin by addition of a 10-fold excess (with respect to the resin) of elution buffer containing 100 μg/ml 3×FLAG peptide (SIGMA). The concentration of purified HDAC is determined by Western blot analysis.

1. A compound according to Formula I: 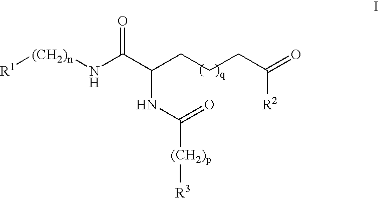
wherein:
a is 0 or 1; b is 0 or 1; m is 0, 1 or 2; n is 0, 1, 2 or 3; p is 0, 1, 2 or 3; and q is 1, 2, 3 or 4;
R¹ is selected from: (C═O)ₐO_{b}(C₁-C₆)alkyl, NH(C═O)(C₁-C₆)alkyl, N(R^{c})₂, (O)ₐ-aryl, (C₃-C₈)cycloalkyl, and heterocyclyl; said alkyl, cycloalkyl, aryl and heterocyclyl optionally substituted with up to three substituents selected from R^{d};
R² is selected from: OH, O(C₁-C₆)alkyl and N(R^{b})₂;
R³ is selected from: H, CF₃, oxo, OH, halogen, CN, N(R^{c})₂, NO₂, (C═O)ₐO_{b}(C₁-C₁₀)alkyl, (C═O)ₐO_{b}(C₂-C₁₀)alkenyl, (C═O)ₐO_{b}(C₂-C₁₀)alkynyl, (C═O)ₐO_{b}(C₃-C₈)cycloalkyl, (C═O)ₐO_{b}(C₁-C₆)alkylene-aryl, (C═O)ₐO_{b}-aryl, (C═O)ₐO_{b}(C₁-C₆)alkylene-heterocyclyl, (C═O)ₐO_{b}-heterocyclyl, NH(C═O)ₐ-aryl, (C₁-C₆)alkyl(O)ₐ-phenyl, (C═O)ₐO_{b}(C₁-C₆)alkylene-N(R^{a})₂, N(R^{a})₂, O_{b}(C₁-C₃)perfluoroalkyl, (C₁-C₆)alkylene-S(O)ₘR^{a}, S(O)ₘR^{a}, C(O)R^{a}, (C₁-C₆)alkylene-CO₂R^{a}, CO₂R^{a}, C(O)H, C(O)N(R^{a})₂, and S(O)₂N(R^{a})₂; said alkyl, alkenyl, alkynyl, cycloalkyl, phenyl, aryl, alkylene and heterocyclyl is optionally substituted with up to three substituents selected from R^{e};
R^{a} is independently selected from: H, oxo, OH, halogen, CO₂H, CN, (O)C═O(C₁-C₆)alkyl, N(R^{c})₂, (C₁-C₆)alkyl, aryl, heterocyclyl, (C₃-C₈)cycloalkyl, (C═O)O(C₁-C₆)alkyl, C═O(C₁-C₆)alkyl and S(O)₂R^{a}; said alkyl, cycloalkyl, aryl or heterocylyl is optionally substituted with one or more substituents selected from OH, (C₁-C₆)alkyl, (C₁-C₆)alkoxy, halogen, CO₂H, CN, (O)C═O(C₁-C₆)alkyl, oxo, N(R^{c})₂ and optionally substituted heterocyclyl, wherein said heterocyclyl is optionally substituted with (C₁-C₆)alkyl, oxo or NH₂;
R^{b} is independently selected from: H, OH, Oₐ(C₁-C₆)alkyl, N(R^{c})₂ and phenyl; said alkyl and phenyl is optionally substituted with phenyl and N(R^{g})₂;
R^{c} is independently selected from: H, (C═O)ₐO_{b}(C₁-C₆)alkyl-phenyl and (C═O)ₐO_{b}(C₁-C₆)alkyl;
R^{d} is independently selected from: NO₂, Oₐ-aryl, Oₐ-heterocyclyl, NH(C═O)-aryl, NH(C═O)(C₁-C₆)alkyl, (C═O)N(R^{c})₂, Oₐ-perfluoroalkyl, OₐCF₃, (C═O)ₐ(C₁-C₆)alkyl, NHS(O)ₘ-aryl, NHS(O)ₘ(C₁-C₆)alkyl, N(R^{c})₂, Oₐ(C₁-C₆)alkyl-heterocyclyl, Oₐ(C₁-C₆)alkyl-N(R^{g})₂, S(O)ₘ(C₁-C₆)alkyl, S(O)ₘ-aryl, (C═O)ₐ-aryl, Oₐ(C₁-C₆)alkyl, CN, S(O)ₘN(R^{c})₂, oxo, OH and halo; wherein said alkyl, aryl and heterocyclyl are optionally substituted with R^{f};
R^{e} is independently selected from: (C═O)ₐCF₃, oxo, OH, halogen, CN, N(R^{c})₂, NO₂, (C═O)ₐO_{b}(C₁-C₁₀)alkyl, (C═O)ₐO_{b}(C₂-C₁₀)alkenyl, (C═O)ₐO_{b}(C₂-C₁₀)alkynyl, (C═O)ₐO_{b}(C₃-C₈)cycloalkyl, (C═O)ₐO_{b}(C₁-C₆)alkylene-aryl, (C═O)ₐO_{b}-aryl, (C═O)ₐO_{b}(C₁-C₆)alkylene-heterocyclyl, (C═O)ₐO_{b}-heterocyclyl, NH(C═O)ₐ-aryl, (C₁-C₆)alkyl(O)ₐ-phenyl, (C═O)ₐO_{b}(C₁-C₆)alkylene-N(R^{a})₂, N(R^{a})₂, O_{b}(C₁-C₃)perfluoroalkyl, (C₁-C₆)alkylene-S(O)ₘR^{a}, S(O)ₘR^{a}, C(O)R^{a}, (C₁-C₆)alkylene-CO₂R^{a}, CO₂R^{a}, C(O)H, (C₁-C₆)alkylₐNH(C₁-C₆)alkyl-N(R^{c})₂, C(O)N(R^{a})₂, and S(O)₂N(R^{a})₂;
R^{f} is independently selected from phenyl, heterocyclyl and Oₐ(C₁-C₆)alkyl;
R^{g} is independently selected from H and (C₁-C₆)alkyl;
or a pharmaceutically acceptable salt or stereoisomer thereof.

2. The compound according to claim 1 of the Formula II; 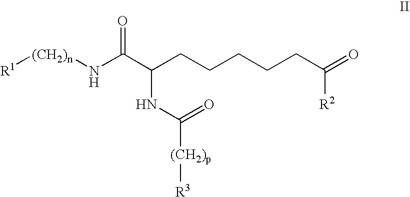
wherein:
all substituents and variables are as defined in claim 1;
or a pharmaceutically acceptable salt or stereoisomer thereof.

3. The compound according to claim 2 of the Formula II;
wherein:
R³ is selected from: H, CN, CF₃, N(R^{c})₂, (C₂-C₁₀)alkenyl, (C₃-C₈)cycloalkyl, S(O)₂(C₁-C₆)alkyl, (C═O)ₐO_{b}(C₁-C₁₀)alkyl, (C═O)ₐ-aryl, (C═O)ₐ-heterocyclyl, S-aryl, S-heterocyclyl, NH(C═O)ₐ-aryl, (C₁-C₆)alkyl(O)ₐ-phenyl; said alkyl, alkenyl, cycloalkyl, aryl and heterocyclyl is optionally substituted with up to three substituents selected from R^{e};
R^{d} is independently selected from: (C═O)ₐ-phenyl, (C₁-C₆)alkylₐ-heterocyclyl, Oₐ(C₁-C₆)alkyl, oxo, CN, S(O)ₘN(R^{c})₂, OH and halo; wherein said alkyl, phenyl and heterocyclyl are optionally substituted with R^{f};
R^{e} is independently selected from: (C═O)ₐ—CF₃, oxo, OH, halogen, CN, N(R^{c})₂, S(O)₂(C₁-C₆)alkyl, (C₁-C₆)alkylₐ(C═O)NH(C₁-C₆)alkyl-N(R^{c})₂, O(C₁-C₆)alkyl-N(R^{c})₂, (C═O)ₐO_{b}(C₁-C₁₀)alkyl, (C₁-C₆)alkyl-phenyl, aryl, heterocyclyl and S(O)₂-phenyl;
and all substituents and variables are as defined in claim 2;
or a pharmaceutically acceptable salt or stereoisomer thereof.

4. A compound which is selected from:
(7S)-7-{[(5-Methoxy-2-methyl-1H-indol-3-yl)acetyl]amino}-8-oxo-8-{[2-(2-phenyl-1H-indol-3-yl)ethyl]amino}octanoic acid;
(2S)-N⁸-(Benzyloxy)-2-{[(5-methoxy-2-methyl-1H-indol-3-yl)acetyl]amino}-N¹-[2-(2-phenyl-1H-indol-3-yl)ethyl]octanediamide;
(2S)-N⁸-(2-Aminophenyl)-2-{[(5-methoxy-2-methyl-1H-indol-3-yl)acetyl]amino}-N¹-[2-(2-phenyl-1H-indol-3-yl)ethyl]octanediamide;
Methyl (7S)-7-{[(5-methoxy-2-methyl-1H-indol-3-yl)acetyl]amino}-8-oxo-8-{[2-(2-phenyl-1H-indol-3-yl)ethyl]amino}octanoate;
(2S)-N⁸-Hydroxy-2-{[(5-methoxy-2-methyl-1H-indol-3-yl)acetyl]amino}-N¹-[2-(2-phenyl-1H-indol-3-yl)ethyl]octanediamide;
(2S)-N⁸-Methoxy-2-{[(5-methoxy-2-methyl-1H-indol-3-yl)acetyl]amino}-N⁸-methyl-N¹-[2-(2-phenyl-1H-indol-3-yl)ethyl]octanediamide;
(2S)-2-{[(5-Methoxy-2-methyl-1H-indol-3-yl)acetyl]amino}-N¹-[2-(2-phenyl-1H-indol-3-yl)ethyl]octanediamide;
(2S)-N⁸-Hydroxy-2-{[(5-methoxy-2-methyl-1H-indol-3-yl)acetyl]amino}-N⁸-methyl-N¹-[2-(2-phenyl-1H-indol-3-yl)ethyl]octanediamide;
(2S)-N⁸-Methoxy-2-{[(5-methoxy-2-methyl-1H-indol-3-yl)acetyl]amino}-N¹-[2-(2-phenyl-1H-indol-3-yl)ethyl]octanediamide;
(2S)-N⁸-Ethoxy-2-{[(5-methoxy-2-methyl-1H-indol-3-yl)acetyl]amino}-N¹-[2-(2-phenyl-1H-indol-3-yl)ethyl]octanediamide;
(2S)-N⁸-(tert-Butoxy)-2-{[(5-methoxy-2-methyl-1H-indol-3-yl)acetyl]amino}-N¹-[2-(2-phenyl-1H-indol-3-yl)ethyl]octanediamide;
(2S)-2-{[(5-Methoxy-2-methyl-1H-indol-3-yl)acetyl]amino}-N⁸-methyl-N¹-[2-(2-phenyl-1H-indol-3-yl)ethyl]octanediamide;
(2S)-2-{[(5-Methoxy-2-methyl-1H-indol-3-yl)acetyl]amino}-N⁸,N⁸-dimethyl-N¹-[2-(2-phenyl-1H-indol-3-yl)ethyl]octanediamide;
(2S)-8-(2,2-Dimethylhydrazino)-2-{[(5-methoxy-2-methyl-1H-indol-3-yl)acetyl]amino}-8-oxo-N-[2-(2-phenyl-1H-indol-3-yl)ethyl]octanamide;
(2S)-N⁸-Benzyl-2-{[(5-methoxy-2-methyl-1H-indol-3-yl)acetyl]amino}-N¹-[2-(2-phenyl-1H-indol-3-yl)ethyl]octanediamide;
(2S)-2-{[(5-Methoxy-2-methyl-1H-indol-3-yl)acetyl]amino}-N⁸-(2-phenylethyl)-N¹-[2-(2-phenyl-1H-indol-3-yl)ethyl]octanediamide;
(2S)-N¹-(4-Chlorophenyl)-N⁸-methoxy-2-{[(5-methoxy-2-methyl-1H-indol-3-yl)acetyl]amino}octanediamide;
(2S)-N¹-(4-Chlorophenyl)-N⁸-hydroxy-2-{[(5-methoxy-2-methyl-1H-indol-3-yl)acetyl]amino}-N⁸-methyloctanediamide;
(2S)-N⁸-Methoxy-2-{[(5-methoxy-2-methyl-1H-indol-3-yl)acetyl]amino}-N¹-quinolin-3-yloctanediamide;
(2S)-N⁸-Methoxy-N¹-[2-(2-phenyl-1H-indol-3-yl)ethyl]-2-[(2-thienylcarbonyl)amino]octanediamide;
(2S)-N¹-(4-Chlorophenyl)-N⁸-hydroxy-2-{[(5-methoxy-2-methyl-1H-indol-3-yl)acetyl]amino}octanediamide;
(2S)-N⁸-Hydroxy-2-{[(5-methoxy-2-methyl-1H-indol-3-yl)acetyl]amino}-N¹-quinolin-3-yl octanediamide;
(2S)-N⁸-Hydroxy-N¹-[2-(2-phenyl-1H-indol-3-yl)ethyl]-2-[(2-thienylcarbonyl)amino]octanediamide;
(2S)-N⁸-(2-Aminophenyl)-N¹-(4-chloro phenyl)-2-{[(5-methoxy-2-methyl-1H-indol-3-yl)acetyl]amino}octanediamide; and
(8S)-8-{[(5-Methoxy-2-methyl-1H-indol-3-yl)acetyl]amino}-9-oxo-9-{[2-(2-phenyl-1H-indol-3-yl)ethyl]amino}nonanoic acid;
or a pharmaceutically acceptable salt or stereoisomer thereof.

5. A pharmaceutical composition comprising a compound according to claim 1 or a pharmaceutically acceptable salt thereof and a pharmaceutically acceptable carrier.

6. A compound according to claim 1, or a pharmaceutically acceptable salt thereof for use in a method of treatment of the human or animal body by therapy.

7. (canceled)

8. (canceled)

9. A method of treating or preventing a disease selected from cancer, neurodegenerative diseases, schizophrenia, stroke, restenosis, mental redardation and immune disorders in a subject, which comprises administration to that subject an effective amount of a compound of claim 1 or a pharmaceutically acceptable salt thereof.

10. A method of treating or preventing a disease selected from neurodegenerative diseases, schizophrenia, inflammatory diseases, restenosis, mental retardation and immune disorders in a subject, which comprises administration to that subject an effective amount of a compound of claim 1 or a pharmaceutically acceptable salt or stereoisomer thereof.
